(19) United States
(12) Patent Application Publication
(10) Pub. No.: US 2006/0052674 A1
(43) Pub. Date: Mar. 9, 2006

(54) Software method of determining and treating psychiatric disorders

(76) Inventor: Steven Eisenstein, Farmville, VA (US)

Correspondence Address: National IP Rights Center, LLC, 550 Township Line Road, Suite 400, Blue Bell, PA, 19422, US

(21) Appl. No.: 10/934,649

(22) Filed: Sep. 4, 2004

(51) Int. Cl. A61B 5/00

(52) U.S. Cl. 600/300; 128/920; 128/923

(57) ABSTRACT

A software method of determining and treating psychiatric disorders by prompting a user is disclosed. The method includes the steps of defining the decline; assessing potential contributing factors leading to the decline; connecting the potential contributing factors to the decline; diagnosing the decline; addressing the decline and identifying potential contributing factors, and developing a care plan; ensuring adequate management of medical and medication factors; and addressing psychotropic management.

### FIELD OF THE INVENTION

The present invention relates to psychiatric aids, and in particular to a method of determining and treating psychiatric disorders utilizing a software program.

### BACKGROUND OF THE INVENTION

An unfortunate effect of aging on human beings is that their minds begin to lose some the faculties that they once possessed. The symptoms that of the onset of some of these losses are visible, but there are also hidden symptoms that are more difficult to diagnose. It would therefore be helpful if there were an aid in diagnosing a patient to determine if he or she may have a psychiatric disorder and what stage it might be in. Particularly, it would be advantageous to have a software program that would guide a physician in diagnosing the patient step by step.

U.S. Pat. No. 5,574,828 is directed towards a system utilizing a software program used to write other software application programs for the implementation of guideline applications for use in situations where a qualification decision or next course of action determination must be made. The system uses questions with limited choice answers. Data provided in answer to the questions causes a second program application to be automatically generated based on the answers. The second application then elicits responses in an interactive manner. Qualification decisions and courses of action are suggested as an output of the second application. Means are provided for evaluating the reliability of the suggestions based on consistency of answers and fatigue of the user. Means are also provided for editing either application program.

U.S. Pat. No. 6,267,722 is directed towards Systems and methods for medical diagnosis or risk assessment for a patient. These systems and methods are designed to be employed at the point of care, such as in emergency rooms and operating rooms, or in any situation in which a rapid and accurate result is desired. The systems and methods process patient data, particularly data from point of care diagnostic tests or assays, including immunoassays, electrocardiograms, X-rays and other such tests, and provide an indication of a medical condition or risk or absence thereof. The systems include an instrument for reading or evaluating the test data and software for converting the data into diagnostic or risk assessment information.

U.S. Pat. No. 6,556,987 is directed to an automatic text classification system which extracts words and word sequences from a text or texts to be analyzed. The extracted words and word sequences are compared with training data comprising words and word sequences together with a measure of probability with respect to the plurality of qualities. Each of the plurality of qualities may be represented by an axis whose two end points correspond to mutually exclusive characteristics. Based on the comparison, the texts to be analyzed are then classified in terms of the plurality of qualities. In addition, a fuzzy logic retrieval system and a system for generating the training data are provided.

U.S. Pat. No. 6,640,219 is directed to data files that are categorized in order to facilitate the searching for information. The analysis is performed in order to identify items which may be considered as having high value without actually being directly specified. Occurrences of unspecified candidate items are identified in contexts for a preferred specified category. Occurrences of unspecified candidate items are identified in non-preferred contexts. The preferred occurrences are processed with the non-preferred occurrences for each candidate item in order to select candidate items as being high value items. In the preferred embodiment, data relating to companies is identified without specific company names being defined.

U.S. Pat. No. 6,611,842 is directed towards a computer system that includes a database storing user histories of selected products, and a database associating products with assessments of their content in a number of different categories. The computer system generates user profile data reflecting the underlying characteristics of user preferences by identifying categories and groups of categories corresponding to products in the user histories whose content assessments are one of an extremely high and low evaluation. In the user profile data, larger groups of categories having extremely high or low content evaluations are weighted more heavily than smaller groups of categories and singly identified categories having extremely high or low content evaluations. The generated user profile data can be utilized to provide targeted advertising and/or to automatically select products are identified with similar underlying characteristics of the user preferences. In one example, a television recording apparatus is provided that automatically records television programs based on a correspondence between program profile data associated with the television programs and user profile data that has been generated based on a past history of the user's viewing habits.

U.S. Pat. No. 6,383,135 is directed towards a medical self-screening system and method that allows rapid triage of patient medical problems. An exemplary system includes a computer having a selection device, a display, and an optional printer. A storage device containing one or more databases is coupled to the computer. Triage software runs on the computer that generates and displays a symptom screen display comprising a pictorial image of the body containing selectable regions that may be affected by patient symptoms. The patient selects a generally affected area or region on the displayed anatomical picture of the body using the selection device. Then the triage software displays a subsequent anatomical picture which is an enlarged more detailed view of the affected area. The patient selects a more specific region of the affected area shown in the enlarged view. The triage software then displays symptom selection screens that permit comparison of groups of symptoms experienced by the patient. The selected symptoms and data derived from the one or more databases are processed to determine an appropriate course of action that should be taken by the patient. The appropriate course of action is displayed to the patient.

U.S. Pat. No. 5,980,447 is directed to an interactive multi-media computer system for providing support and guide to an individual undergoing recovery from a substance or emotional dependency. The computer system including a central processing unit, a monitor, user input device and a CD ROM for reading a pre-recorded medium containing interactive programming material. The CD ROM has data recorded on it for implementing computer routines which interactive engage the user and provide a crisis module for interactively testing and evaluating a user's mental condition and recommending specific procedures to come out of adverse mental conditions depending upon the results of the test. The CD ROM also contains a browse module with resource materials which are related to education in the realm of the recovery process and a quest module containing control software for structuring a specific program for the user to follow to further the user's progress in the recovery process.

U.S. Patent Application No. 20030135095 is directed to a system and method for providing computerized, knowledge-based medical diagnostic and treatment advice. The medical advice is provided to the general public over networks, such as a telephone network or a computer network. The invention also includes a stand-alone embodiment that may utilize occasional connectivity to a central computer by use of a network, such as the Internet. New authoring languages, interactive voice response and speech recognition are used to enable expert and general practitioner knowledge to be encoded for access by the public. “Meta” functions for time-density analysis of a number of factors regarding the number of medical complaints per unit of time are an integral part of the system. A re-enter feature monitors the user's changing condition over time. A symptom severity analysis helps to respond to the changing conditions. System sensitivity factors may be changed at a global level or other levels to adjust the system advice as necessary.

U.S. Patent Application No. 20030140928 is directed towards a system and method for providing medical treatment, such as medication, to a patient. The administration of the medication may include the use of an infusion pump. The system may be implemented in a variety of ways including as a computer program. The computer program accessing information related to the identity of a clinician, the identity of a patient, the identity of a medical treatment, and the identity of a medical device. The computer program determines whether the medical treatment has been previously associated with the patient and whether a plurality of operating parameters for the medical device is consistent with the medical treatment. The computer program also includes logic for providing a first error signal if the medical treatment has not been previously identified with the patient; and a second error signal if the operating parameters for the medical device are not consistent with the medical treatment.

None of the above inventions, however, address a need for a software program which, when used by a professional, allows the professional to rapidly, and effectively diagnose a psychiatric disorder. Accordingly, it would be beneficial if a software program existed that allowed for the rapid and effective diagnoses of psychiatric disorders, particularly in the elderly. The program could be easily set up on a computer and would follow a series of questions which a qualified professional would answer. The program would then be able to ask followup questions to hone in the diagnosis.

### OBJECTS AND SUMMARY OF THE INVENTION

It is an object of the present invention to provide a method of determining and treating psychiatric disorders.

It is a further object of the present invention to provide a method of determining and treating psychiatric disorders that includes a simple to use computer program that guides the user through the necessary steps to aid a psychiatric patient.

It is yet a further object of the present invention to provide a software method of determining and treating psychiatric disorders by prompting a user including the steps of defining the decline; assessing potential contributing factors leading to the decline; connecting the potential contributing factors to the decline; diagnosing the decline; addressing the decline and identifying potential contributing factors, and developing a care plan; ensuring adequate management of medical and medication factors; and addressing psychotropic management.

In accordance with a first aspect of the present invention, a novel method of determining and treating psychiatric disorders is disclosed.

In accordance with another aspect of the present invention, a novel software method of determining and treating psychiatric disorders by prompting a user is disclosed. The method includes the steps of defining the decline; assessing potential contributing factors leading to the decline; connecting the potential contributing factors to the decline; diagnosing the decline; addressing the decline and identifying potential contributing factors, and developing a care plan; ensuring adequate management of medical and medication factors; and addressing psychotropic management.

### BRIEF DESCRIPTION OF THE DRAWINGS

The foregoing summary, as well as the following detailed description of a preferred embodiment of the present invention will be better understood when read with reference to the appended drawings, wherein:

FIG. 1 is a flow diagram depicting a typical method of determining and treating psychiatric disorders in accordance with the present invention.

FIG. 2 is a flow diagram of a step of ensuring adequate management of medical and medication factors in accordance with the present invention of FIG. 1.

FIGS. 3-4 are screenshots of start page screens in accordance with the present invention.

FIGS. 5-10 are screenshots of step one of the method of determining and treating psychiatric disorders in accordance with the present invention.

FIGS. 11-24 are screenshots of step two of the method of determining and treating psychiatric disorders in accordance with the present invention.

FIGS. 25-26 are screenshots of step three of the method of determining and treating psychiatric disorders in accordance with the present invention.

FIGS. 27-31 are screenshots of step four of the method of determining and treating psychiatric disorders in accordance with the present invention.

FIGS. 32-36 are screenshots of step five of the method of determining and treating psychiatric disorders in accordance with the present invention.

FIGS. 37-38 are screenshots of step six of the method of determining and treating psychiatric disorders in accordance with the present invention.

FIGS. 39-42 are screenshots of step seven of the method of determining and treating psychiatric disorders in accordance with the present invention.

FIGS. 43-53 are screenshots of administration screens of the method of determining and treating psychiatric disorders in accordance with the present invention.

### DETAILED DESCRIPTION OF THE PREFERRED EMBODIMENT

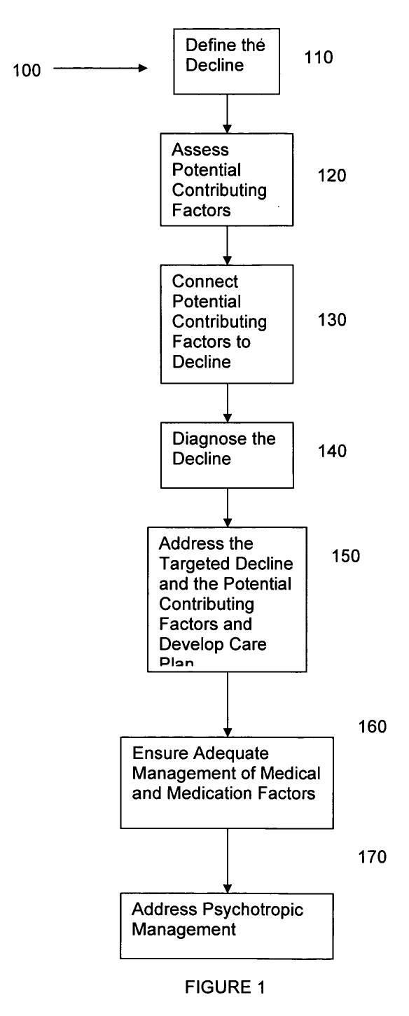

Referring now to the drawings, wherein like reference numerals refer to the same components across the several views and in particular to FIG. 1, a software method of determining and treating psychiatric disorders 100 is shown. The method of determining and treating psychiatric disorders 100 includes a comprehensive seven step assessment and treatment program focusing on functional and behavioral problems, particularly in the elderly.

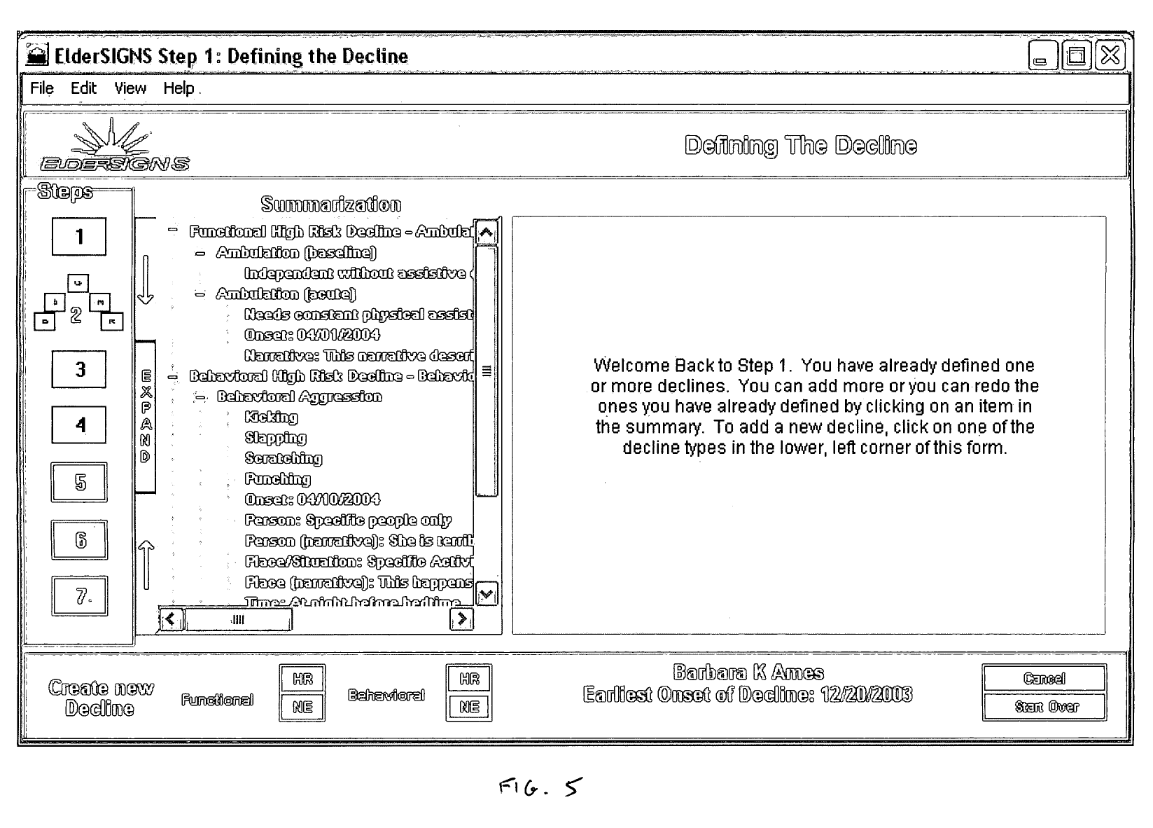

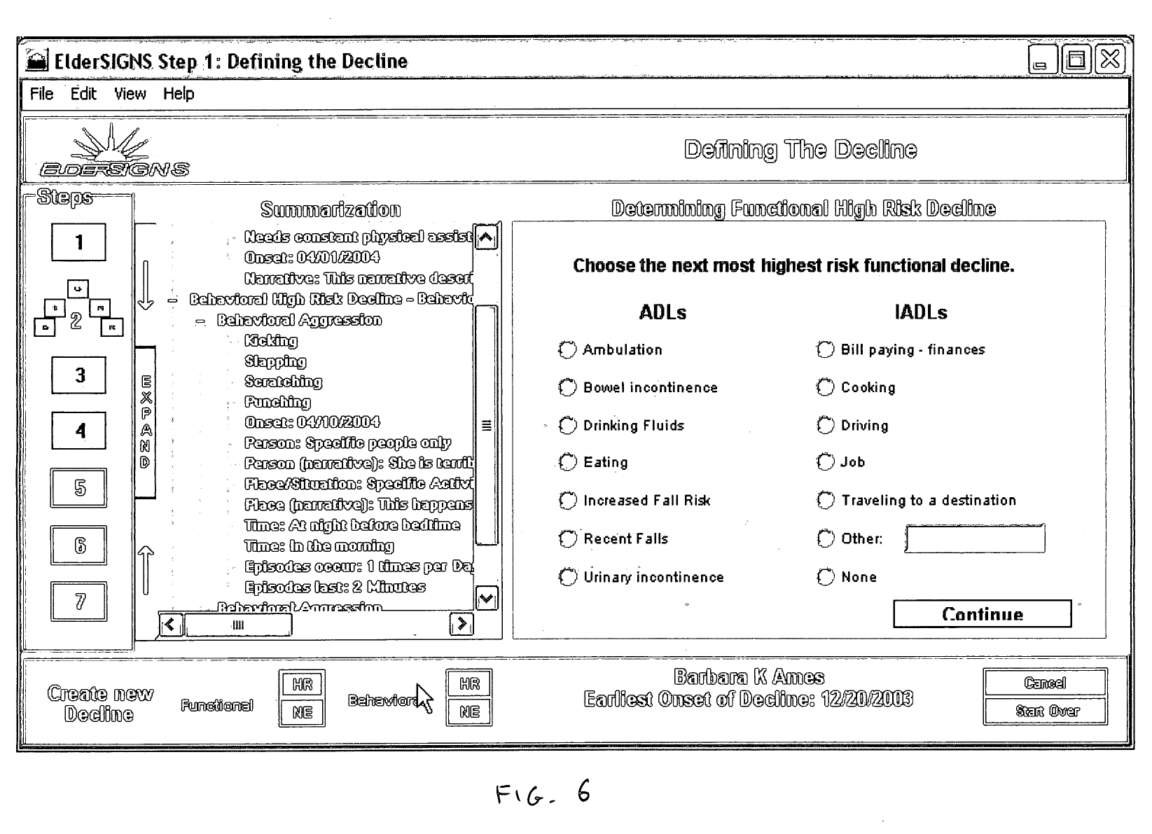

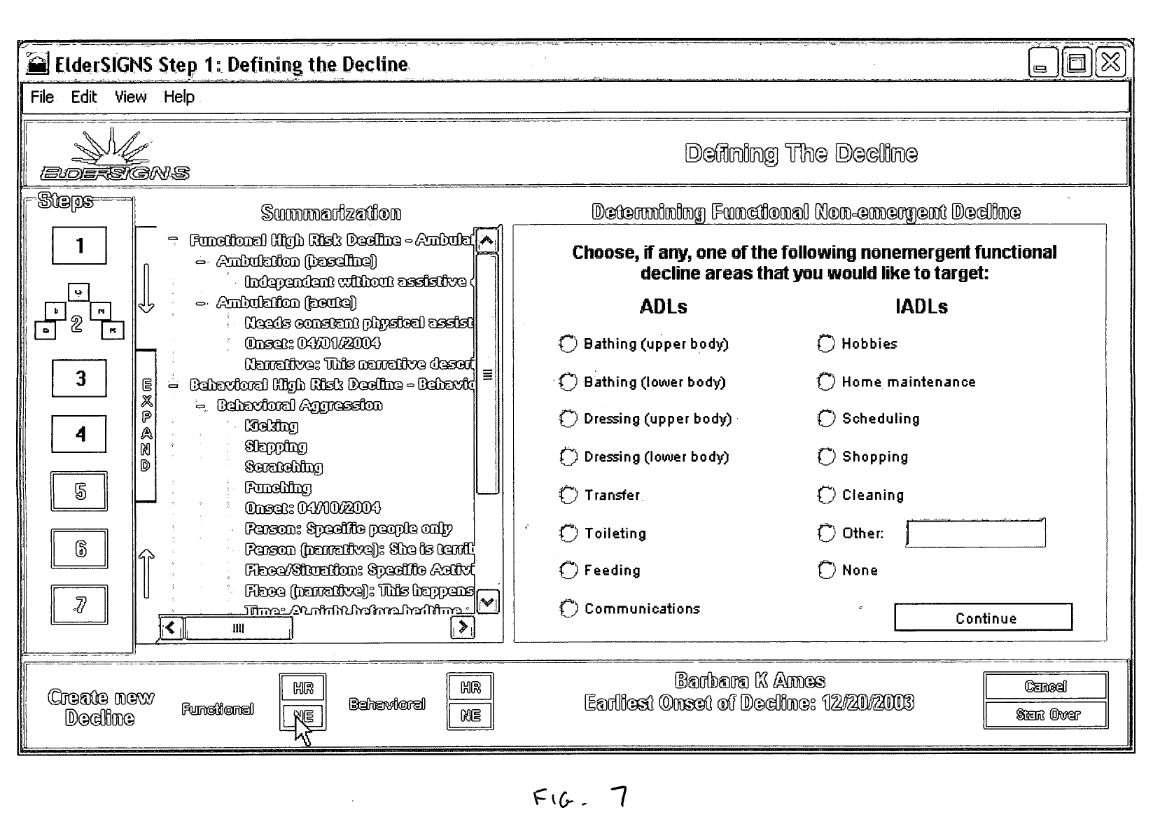

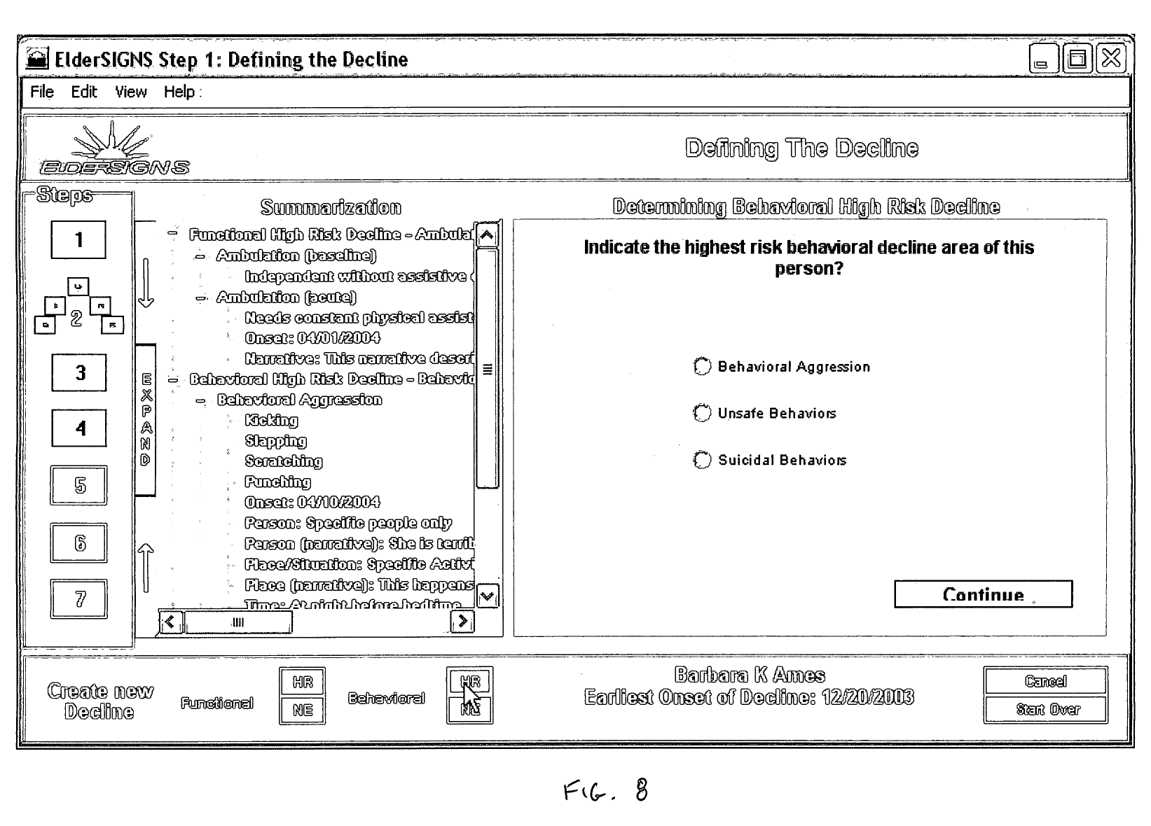

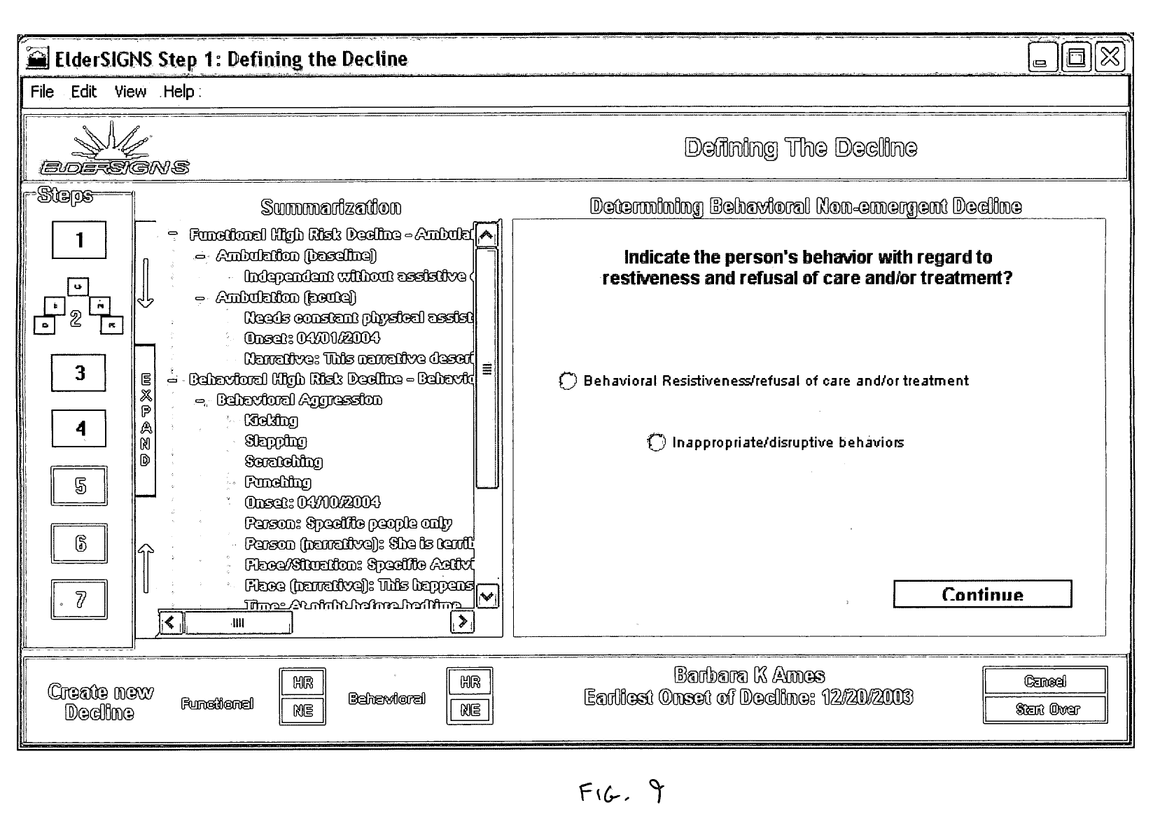

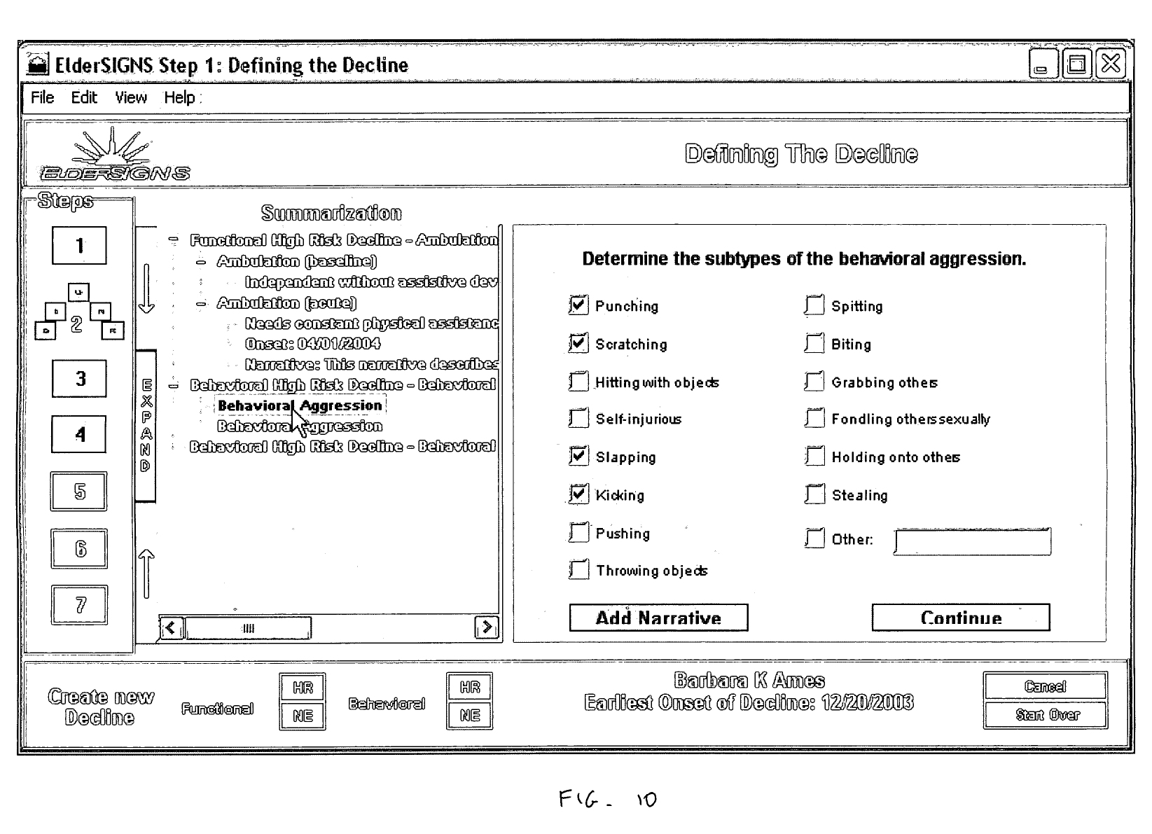

Referring now to FIGS. 1, and 5-10, the first step 110 in the software method of determining and treating psychiatric disorders 100 is defining the decline. The step of defining the decline 110 includes having the user identify his or her patient's decline, especially acute high risk ones. The program prompts a user to prioritize behavioral and functional decline into high-risk and non-emergent and through a series of prompts describes each identified targeted decline in detail using observable and measurable descriptors related to time, situation/place and people involved in the decline. The pre-existing baseline of the acute targeted decline is also delineated which becomes the goal for the care plan of the targeted decline which is developed in step 5.

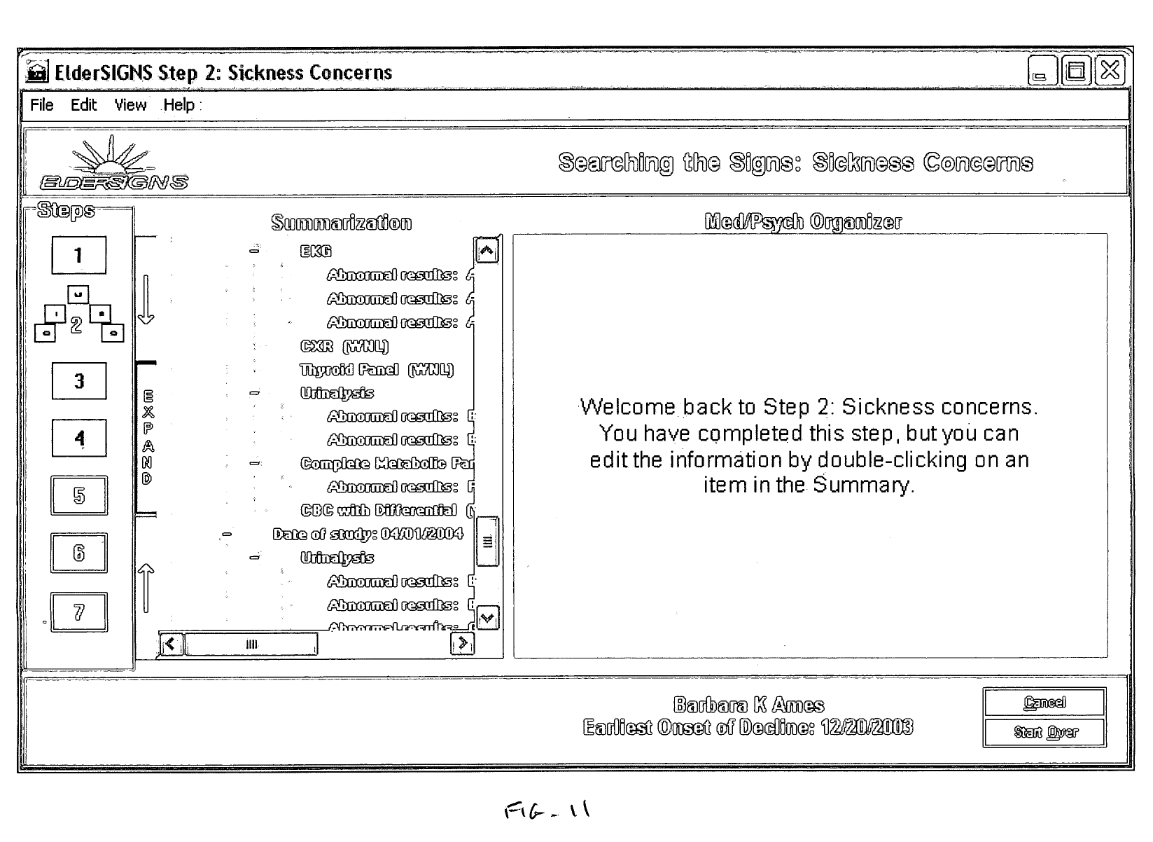

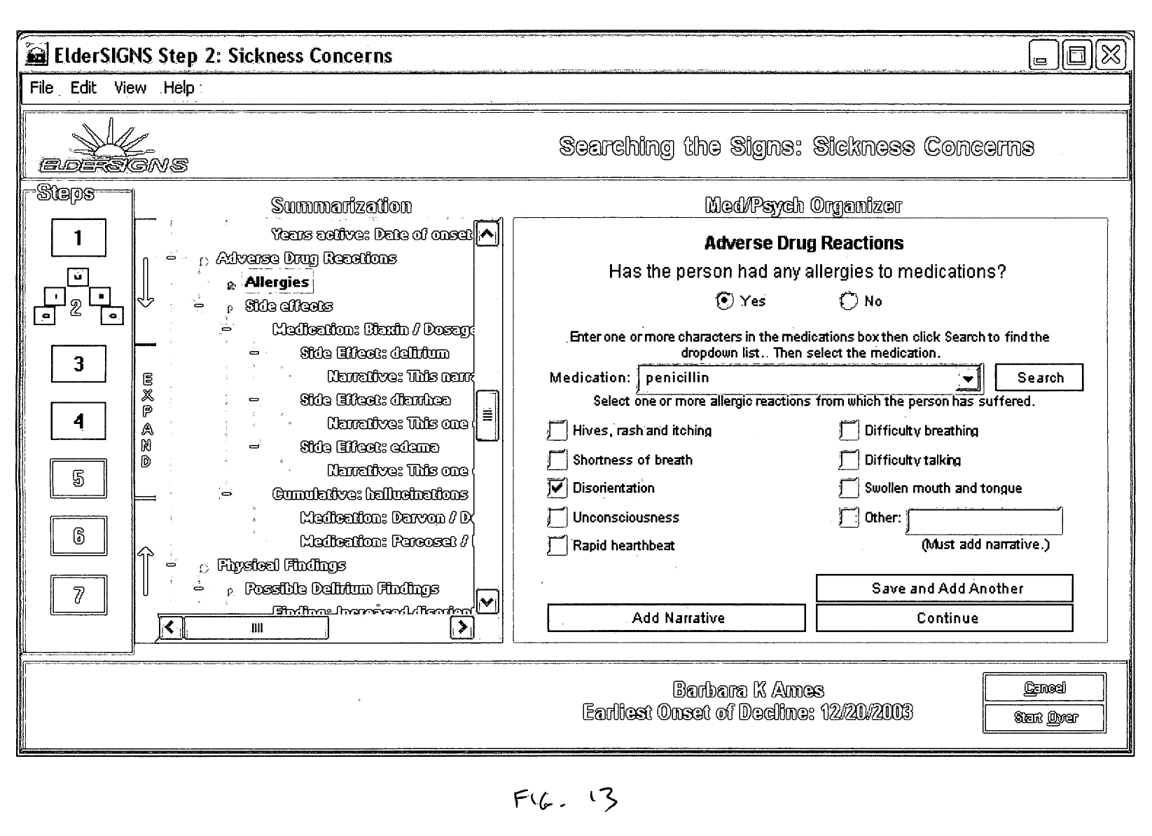

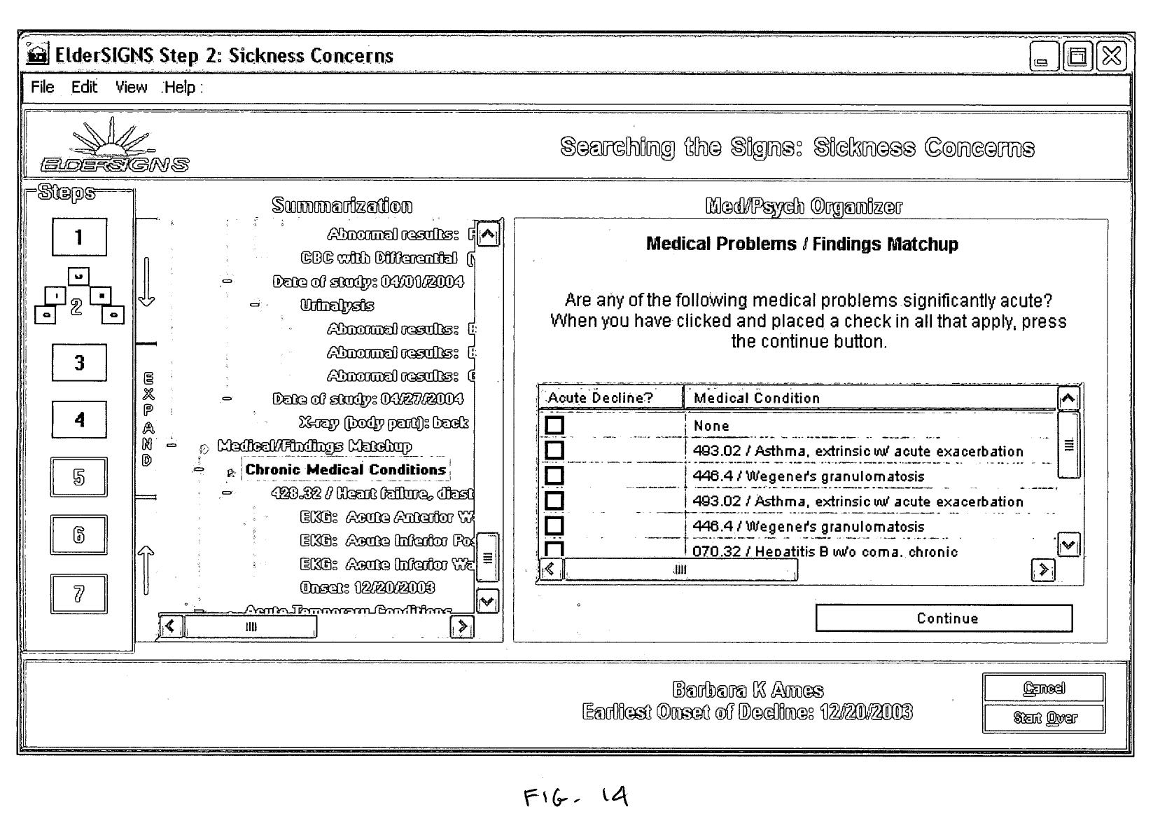

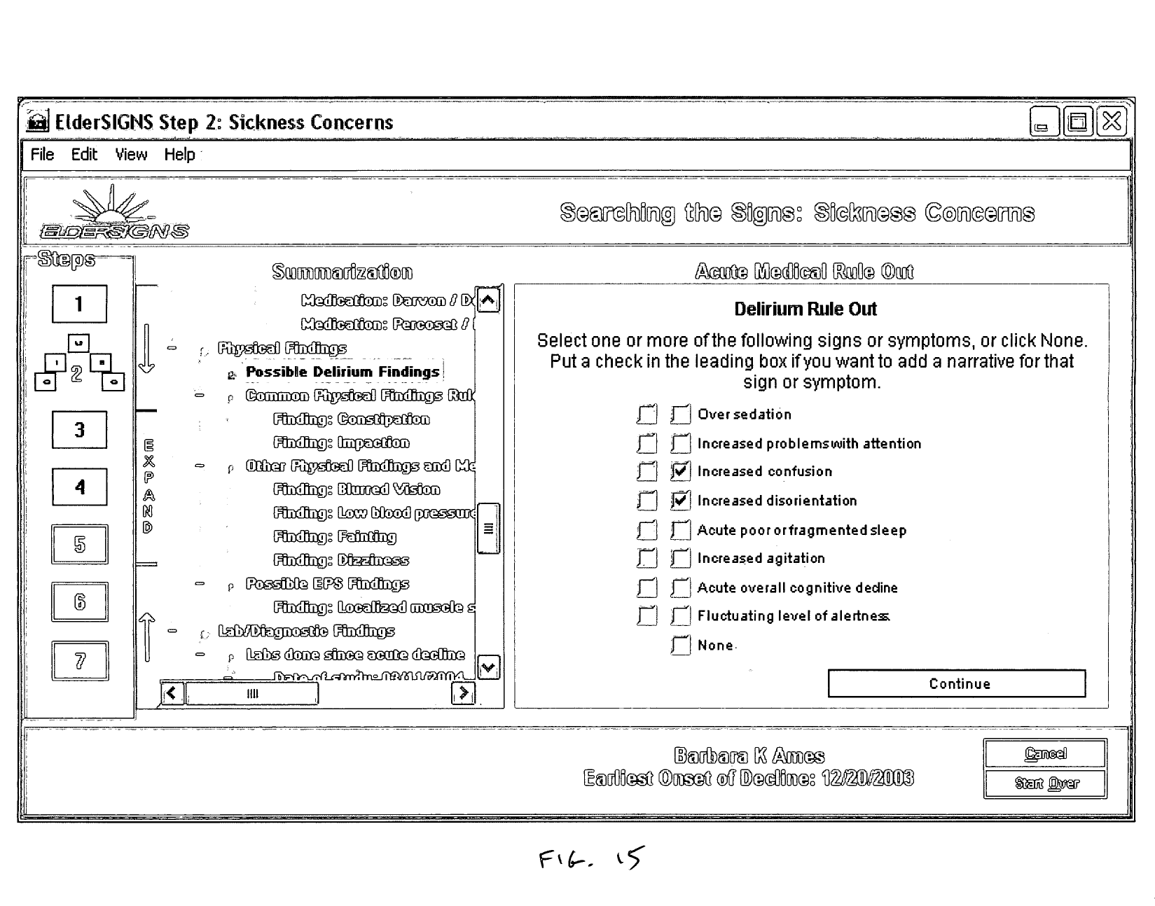

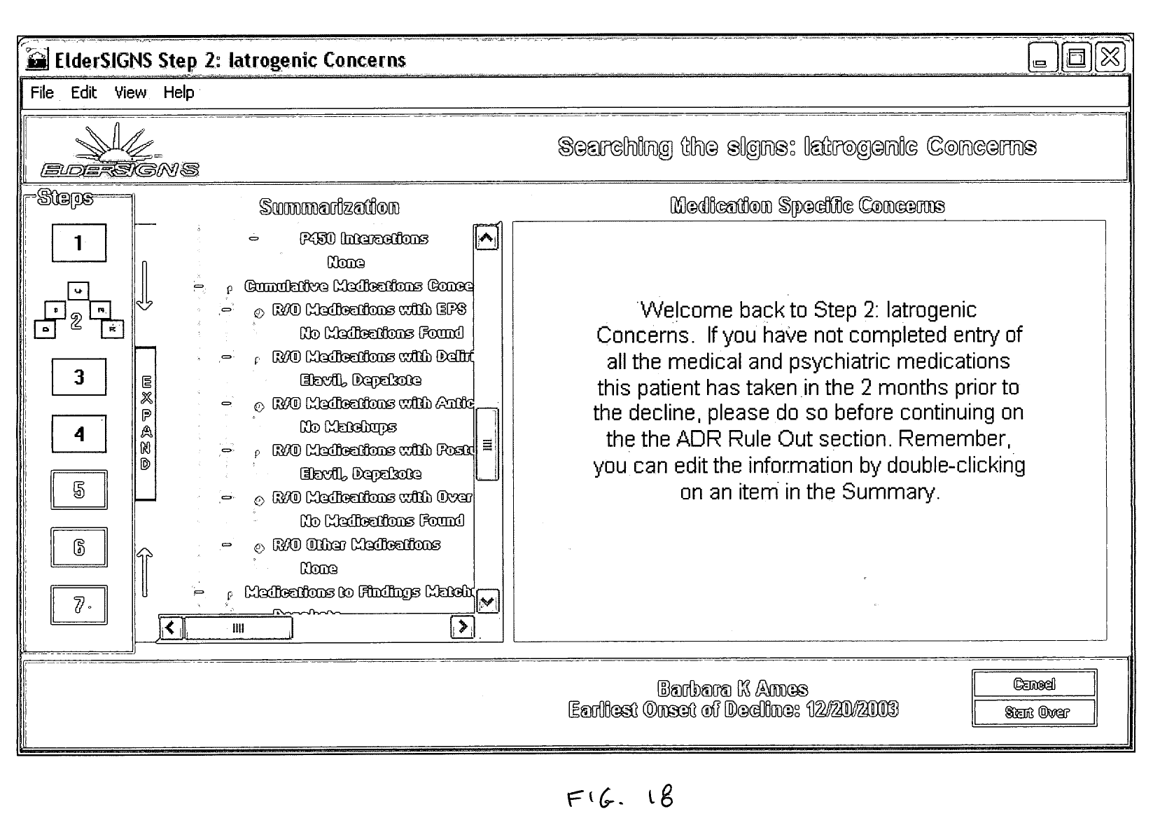

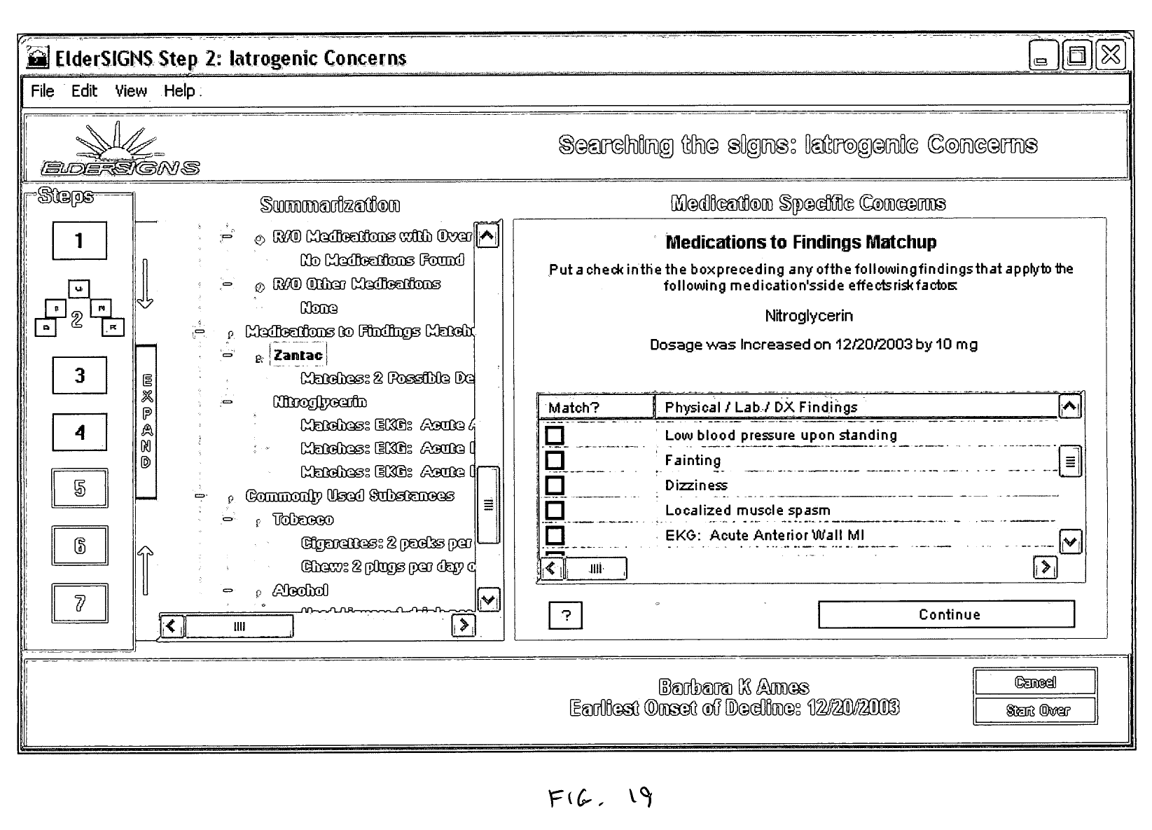

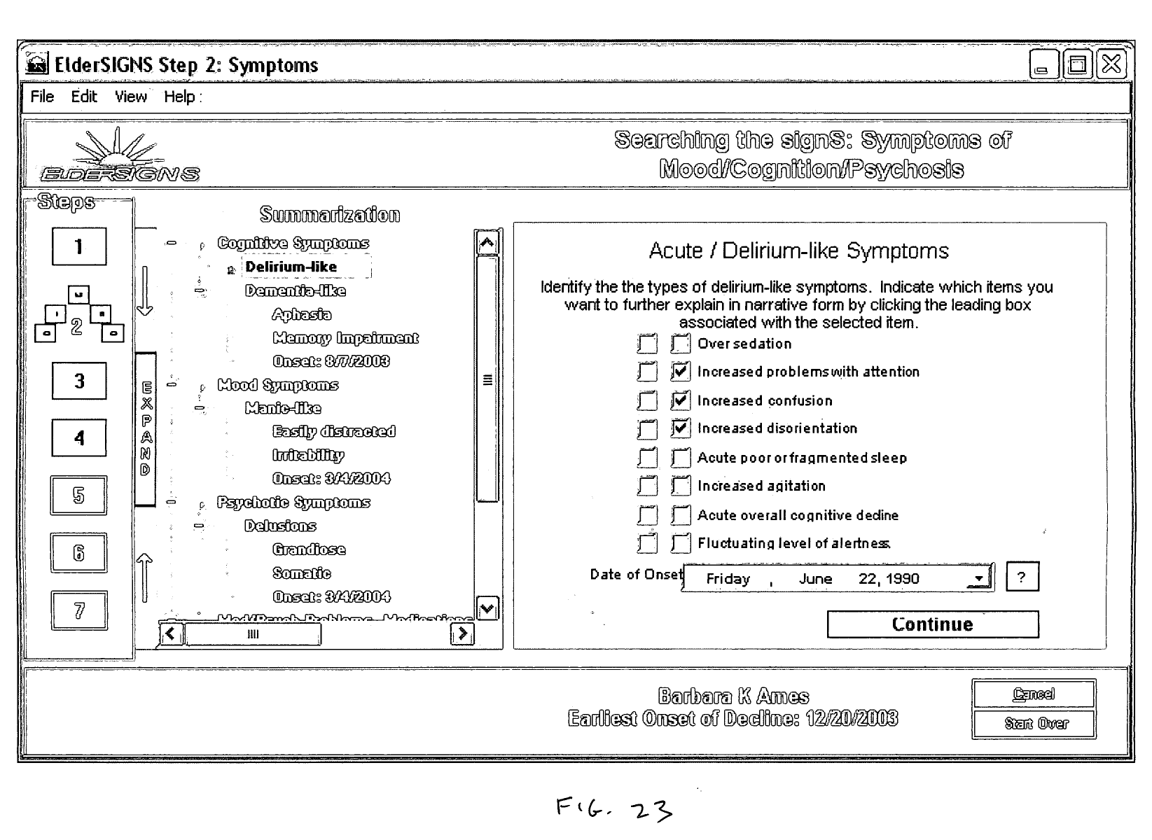

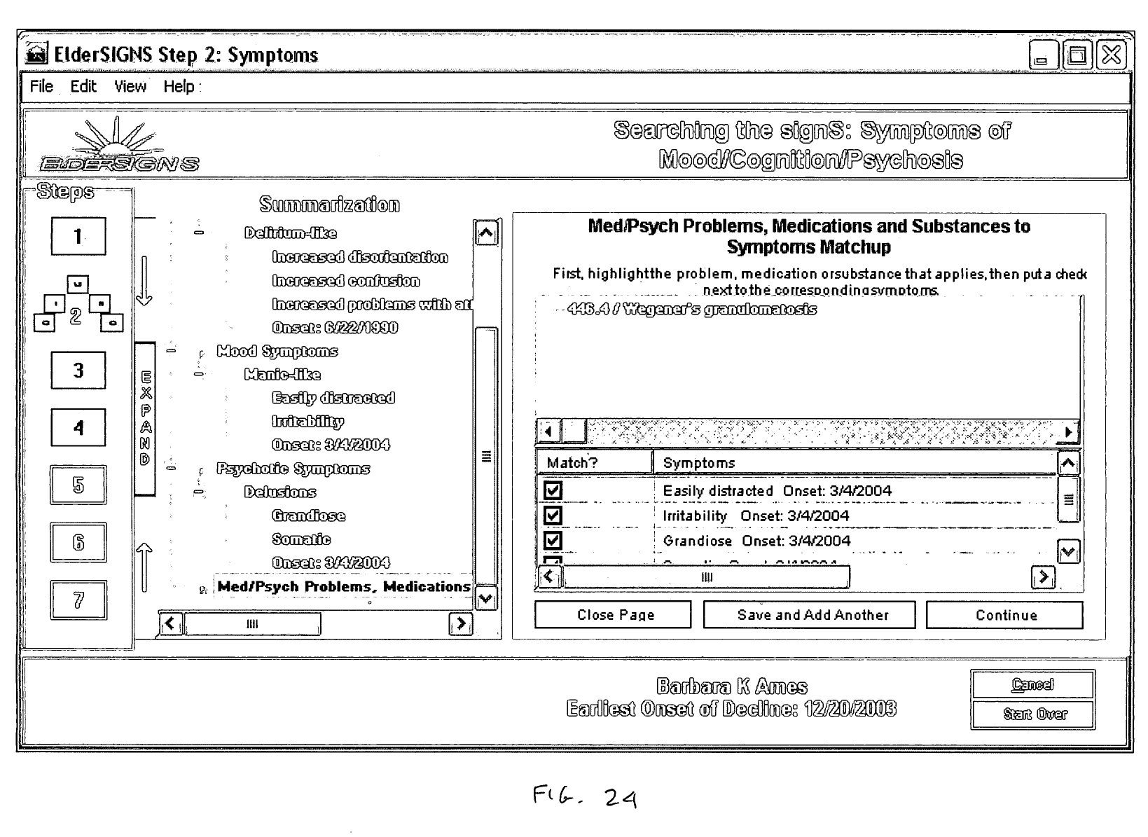

Referring now additionally to FIGS. 11-24 as well as FIG. 1, step two involves assessing the potential contributing factors leading to the targeted decline 120. It is the assessment heart of the software. The user is guided through a series of SIGNS by the software program, with SIGNS being a mnemonic for sickness, iatrogenic (medication) concerns, global functional concerns, nuance-stressors, and symptoms of psychiatric illness. The software program guides the user through each of these important clinical assessment areas and will highlight any potentially contributory factors.

In particular, The Sickness and Iatrogenic Concerns substeps are expert-guided assessments focusing on identifying potentially acute medical and medication problems that could be contributing to the targeted decline/s in Step 1.

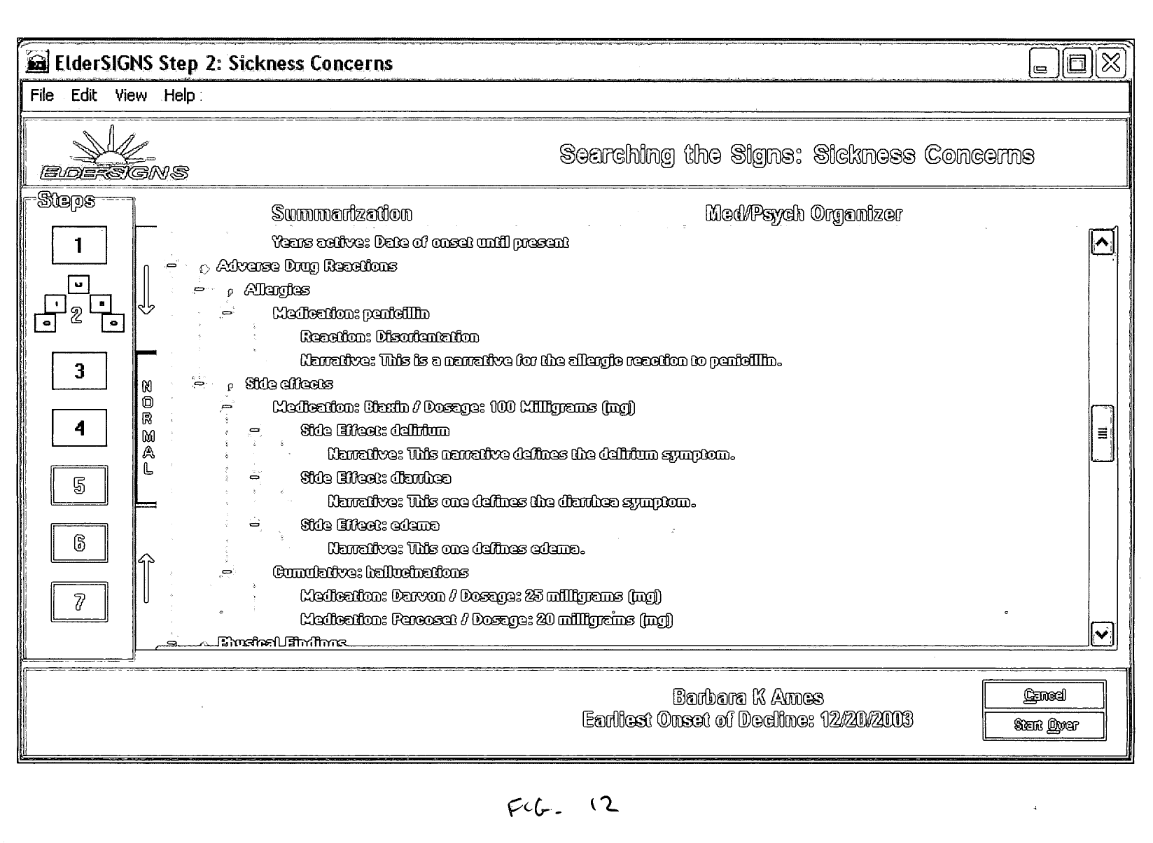

In the Sickness substep, shown in FIGS. 12-15, the user is first structured to complete a full medical and psychiatry history including medical and psychiatric diagnoses along with their ICD-9 codes, identifying certain chronic medical problems that present with repeated behavioral and/or functional decline, a detailed adverse drug reaction summary delineating allergies, medication-specific side effects, and cumulative side effects involving >1 medication at a time. Once the medical history is inputted, the Sickness substep is structured to rule out important acute clinical problems such as delirium, common physical findings, and EPS (extrapyramidal symptoms) and identify all abnormal lab and diagnostic findings as well. The user is then prompted to match-up the identified acute medical findings with any of the person's chronic medical problems. The user is then prompted to match-up any of the identified acute medical findings with any acute temporary medical conditions. All medical concerns i.e. Chronic medical problems with matched acute findings, acute temporary medical problems with matched acute findings, or unmatched acute findings will go to Step 3 for consideration as an active contributor to the targeted decline/s in Step 1.

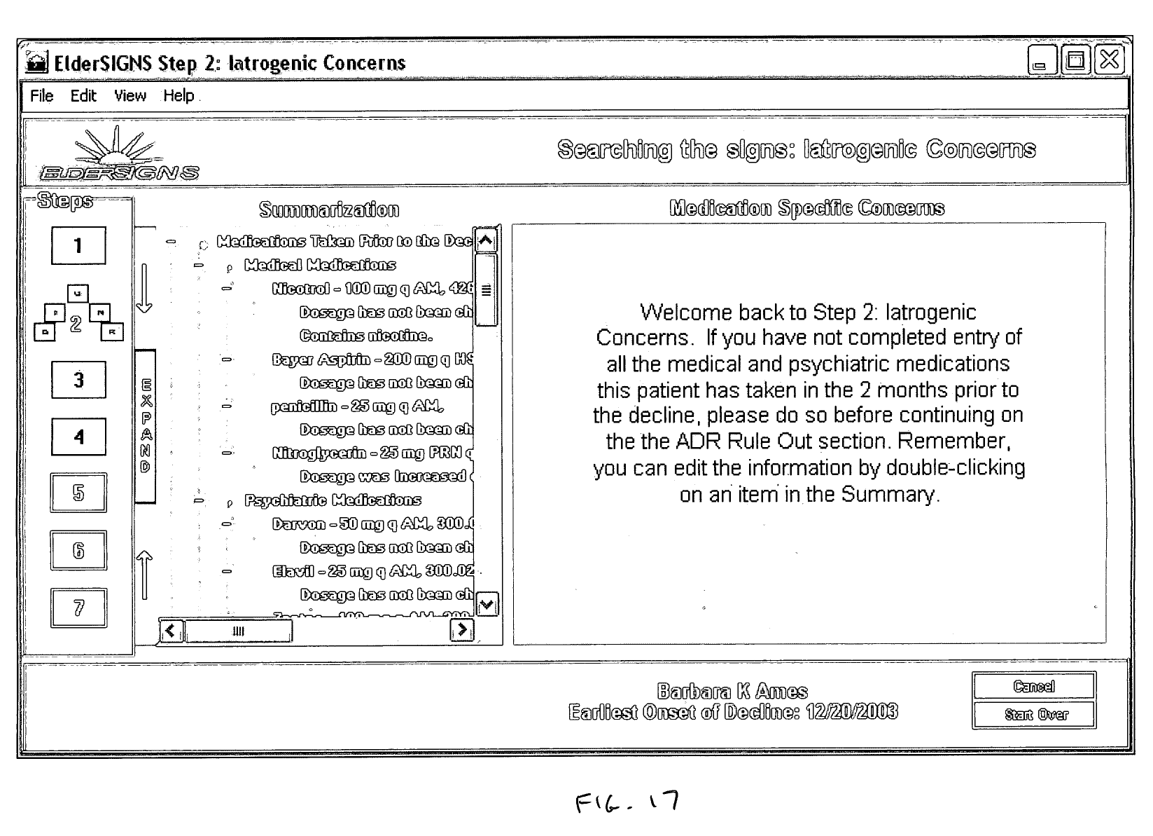

In the Iatrogenic Concerns substep, generally shown in screenshots depicted in FIGS. 17-19, the user is structured to first put in all of the patient's medications being taken before the onset of the decline including ones started or stopped within 6 weeks of the decline. Each medication is chosen from a database of over 18,000 agents including both prescriptions and OTCs including vitamins, dietary supplements, and herbal remedies. For each agent, the user has to associate it with a medical problem, and is prompted to put in the dosage and schedule as well as any medication refusals over the last 7 days prior to the decline and any dosage changes over the six weeks prior to the decline.

Once that is done, the software automatically cross-references the entire medication list to certain medication concern rule outs that have been triggered. These different cumulative side effect lists are triggered to be cross-referenced to the medications when specific associated physical findings are acute and identified on step 1. These cumulative side effects include sedation, anticholinergic toxicity, postural hypotension, EPS, and delirium. Other important medication risk factors that are automatically checked are medications requiring blood levels and P450 drug-drug interactions. All of the potentially identified medication risk factors are then presented in a structured fashion to help the user determine if they are connected to the acute physical findings identified in the Sickness substep. Any matches would then go to Step 3 for consideration as actual contributors to the decline including automatically any medications that have had dosage changes, missed dosages, and no medication blood level drawn since the decline.

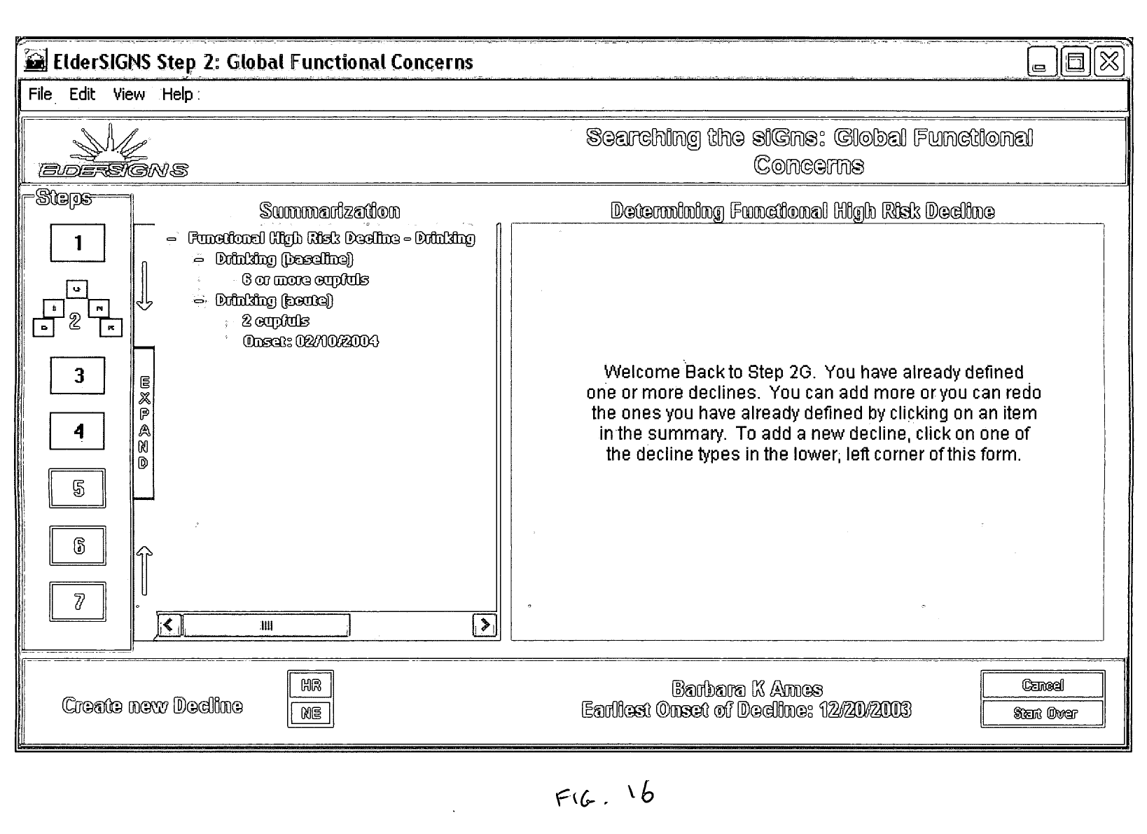

The Global Functional Concerns Substep of Step 2, a screenshot which is depicted in FIG. 16, prompts the user to describe any other functional declines not targeted in Step 1 so that they can be identified and monitored as well to make sure they too resolve as the target declines resolve. Those identified concerns go to Step 5 as well to remind the user if they formally want to monitor its status.

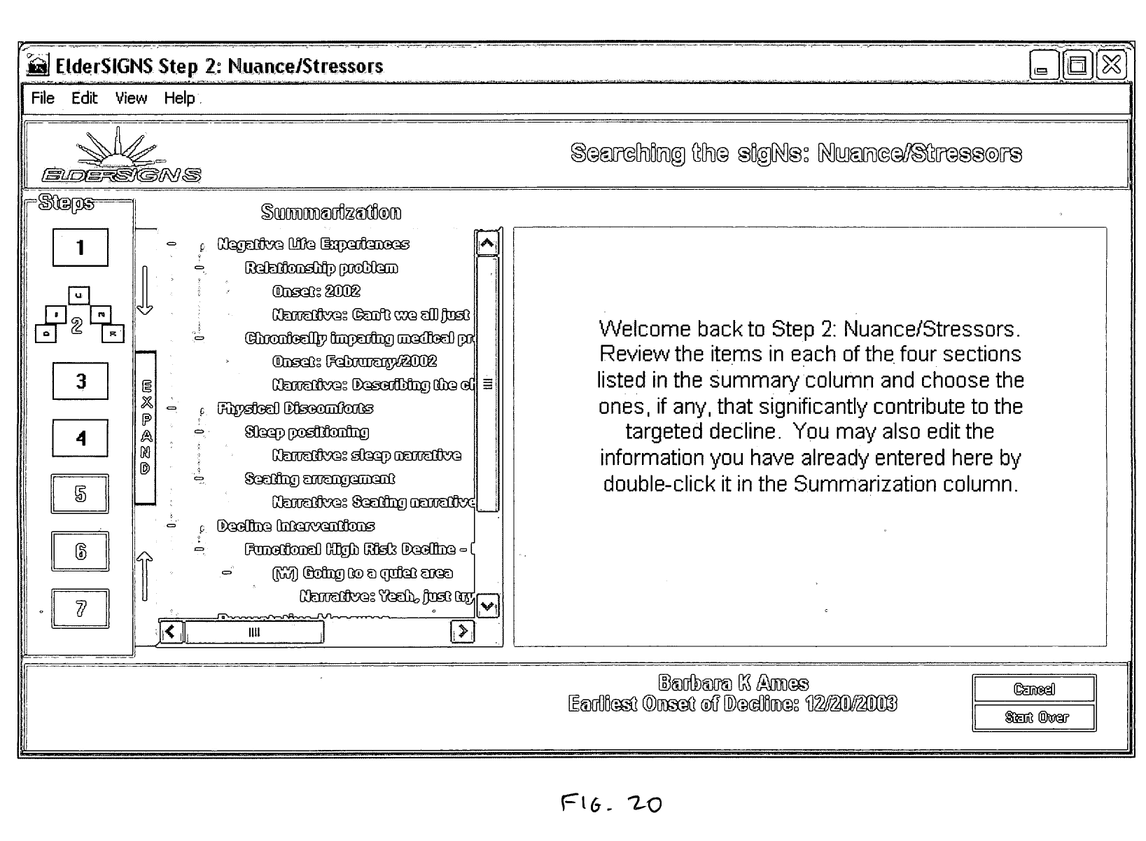

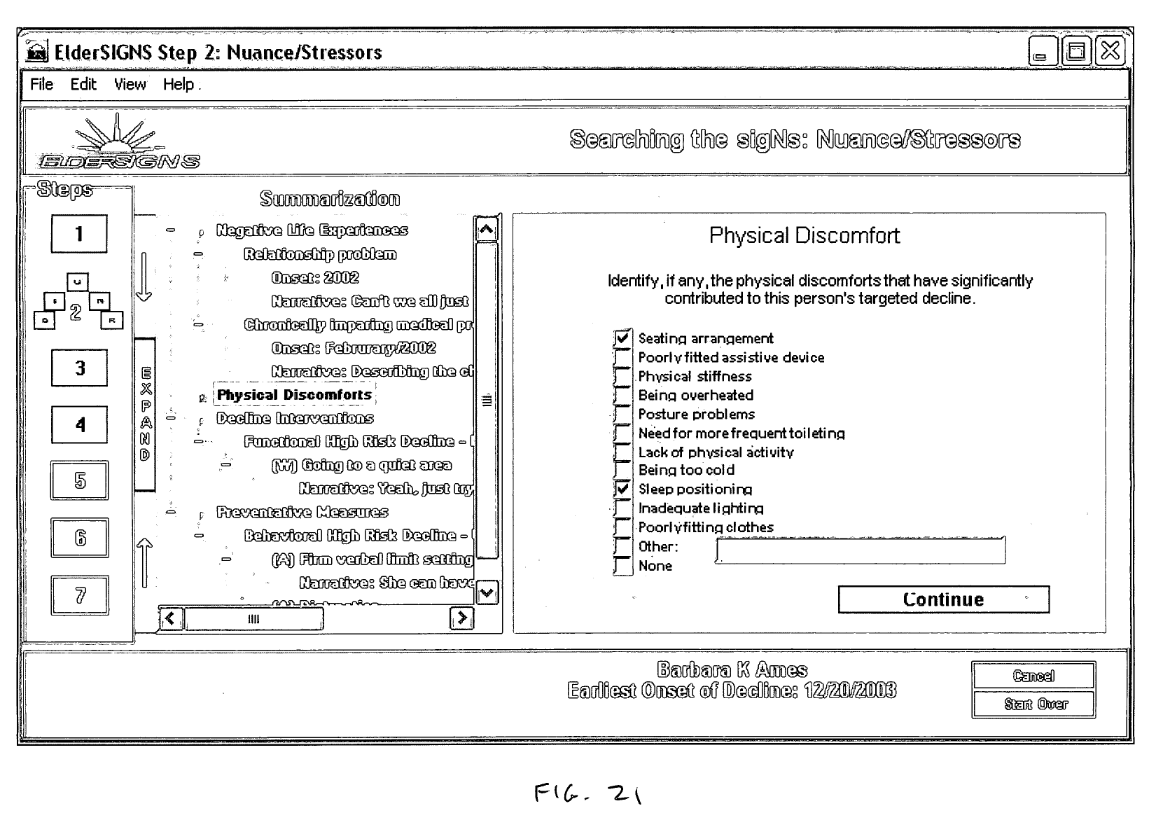

Shown in FIGS. 20-21, Nuance Stressors Substep of Step 2 are all the potential environmental factors that could be impacting on the patient's targeted decline/s. This Substep is available to all clinical team members for them to input pertinent clinical data. The user is prompted to consider a broad range of environmental stressors broken down into four categories: Negative Life Experiences, Physical Discomfort, Previously Tried Decline Interventions, and Previously Tried Preventative Measures of the Decline. Negative Life Experiences and Physical Discomfort factors identified on Step 2 will go to Step 3 for consideration as actual contributors to the decline. Every Previously Tried Intervention or Preventative Measure of the Decline will be prompted by the user to be identified as alleviating or worsening the incidence of Decline and will then automatically go to Step 5 to prompt the users to put them into the care plan either as interventions to prevent or alleviate the decline or as interventions to avoid to prevent further decline.

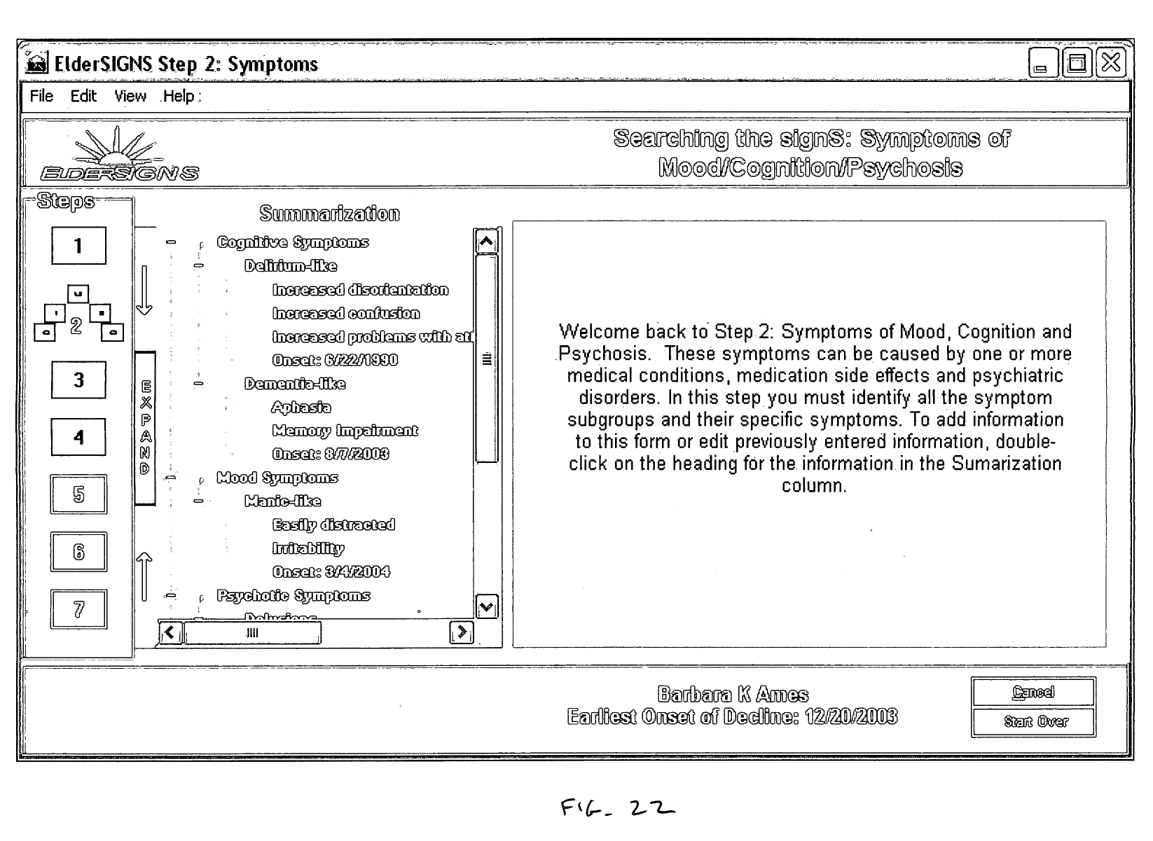

Symptoms of Cognition, Mood, and Psychosis Substep of Step 2 are shown in FIGS. 22-24. This substep structures the user to divide the symptoms of psychiatric illness into three groups: Cognitive, Mood, and Psychotic groups. The Mood Sx group is subdivided into depressive, anxiety-based, and manic-like symptoms. The Cognitive Sx group is subdivided into delirium-like and dementia-like symptoms. Delirium-like symptoms identified under Step 2 Sickness substep will automatically go to the delirium-like symptoms page under Symptoms of Cognition to make sure it is not forgotten, at which time the user can add to them to help fully rule-out delirium. The Psychotic Sx group is subdivided into delusions, hallucinations, and thought disorder. Each subgroup has a list of unique descriptors that the user can click on to identify them as being acute. Once the acute psychiatric symptoms have all been identified, the user is then prompted to match up any of the psychiatric symptoms identified to any of the acute medical conditions and medication concerns identified in the substeps of Sickness and Iatrogenic Concerns respectively.

All matched information as well as unmatched psychiatric symptoms then goes automatically to Step 3 for consideration as active contributors to the decline.

In step three, connecting the potential contributing factors, or SIGNS, to the decline 130, the user will be given a summary of the targeted declines and potential contributory factors along with their respective dates of onset and will use temporal cause and effect and common sense to set up a formulation of specifically chosen contributory factors leading to the functional and or behavioral decline.

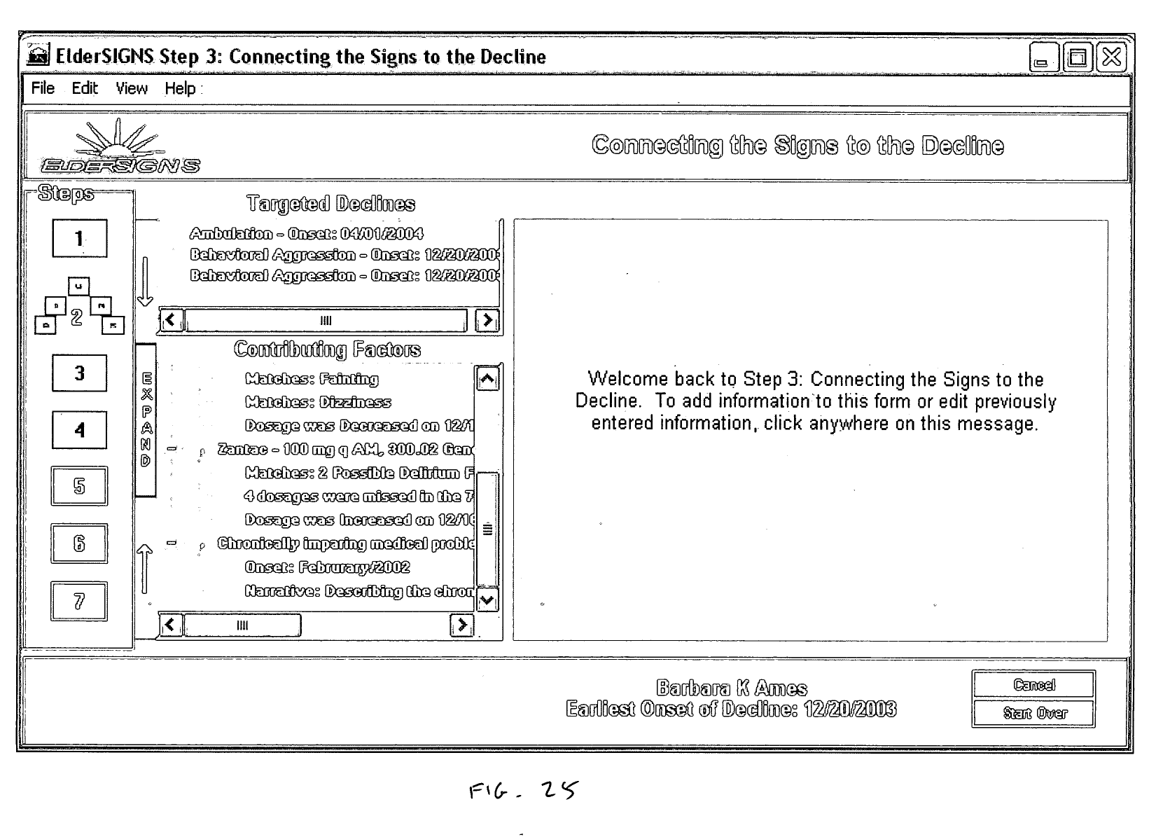

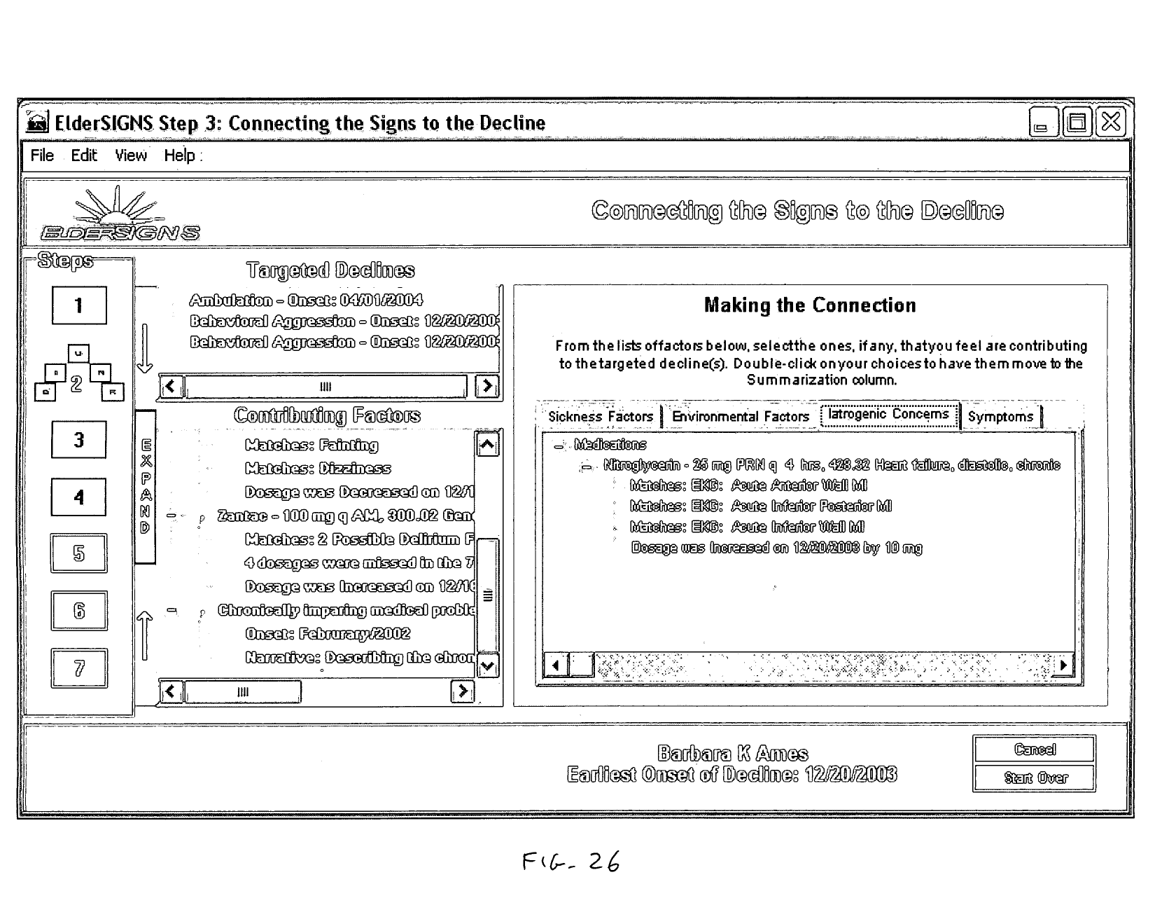

Generally depicted in screenshots in FIGS. 25-26, the software automatically receives all the potential contributing factors identified in the 5 substeps of Step 2. The user then reviews all the potential contributors and then chooses which factors are actually contributory. This step uses the dates of onset of the decline/s as well as the dates of onset of the potential contributory factors to help create cause and effect to facilitate decision-making. All clinical contributory factors then go automatically to Step 5, where the appropriate staff are structured and prompted to treat each one of the contributors to the decline.

Step four involves the psychiatric diagnosis of the decline 140. In step 140, information is taken from the previous three steps and the software program asks a series of questions to the user to help determine how many psychiatric disorders are active, if the active psychiatric disorders are medically based to diagnose them, and if the psychiatric disorders are not medically based, then the software program guides the user to determine the appropriate strictly psychiatric diagnosis.

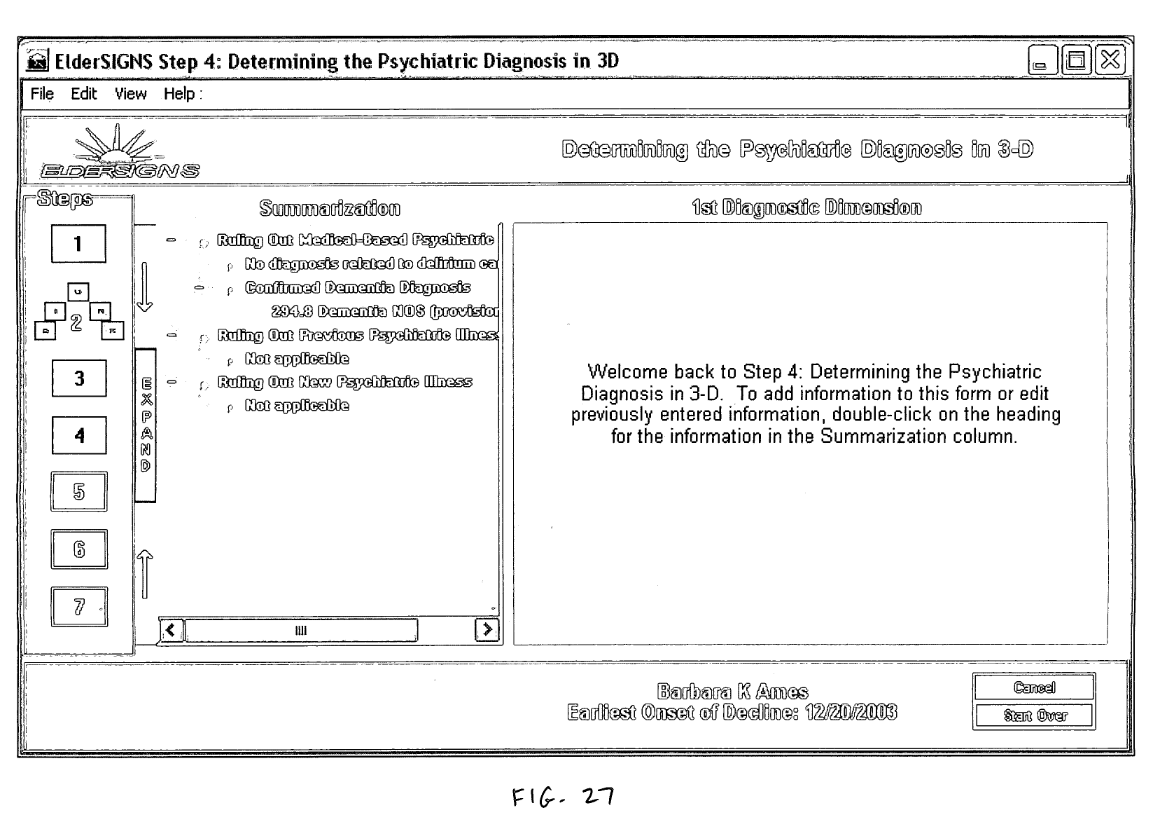

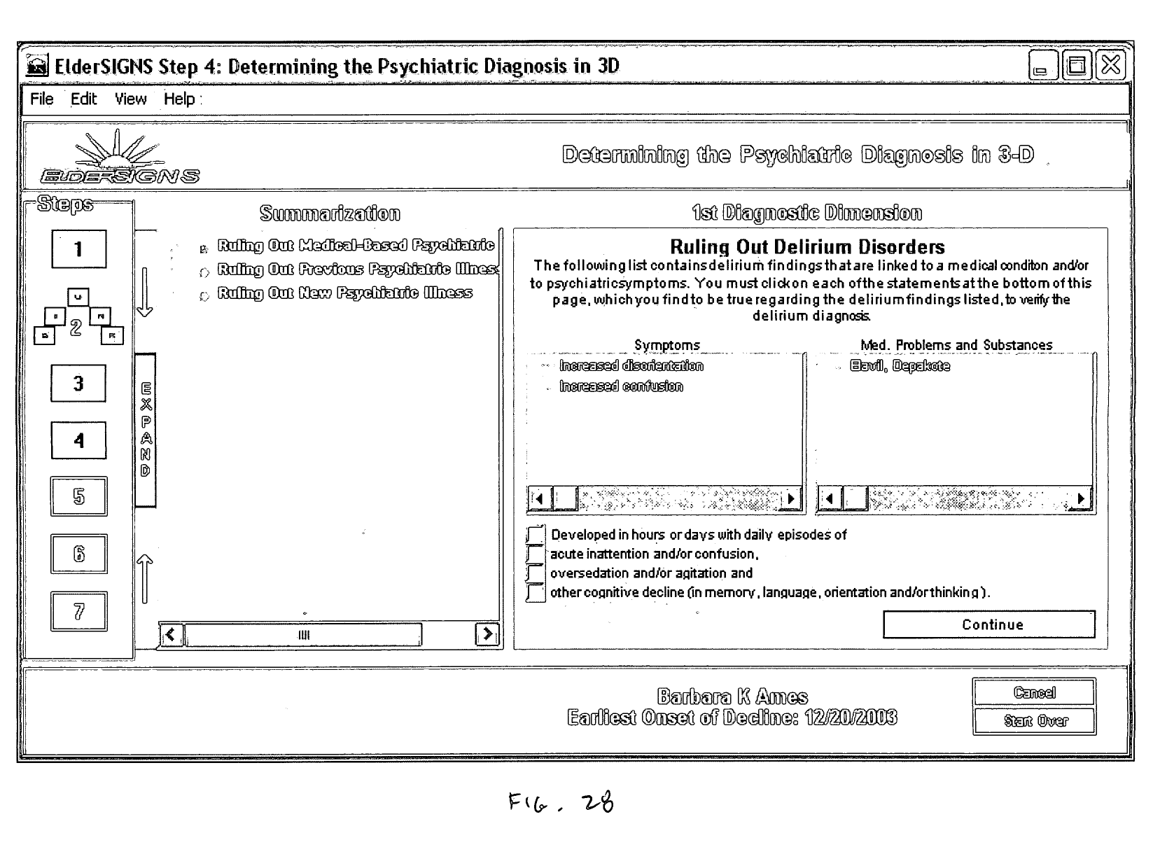

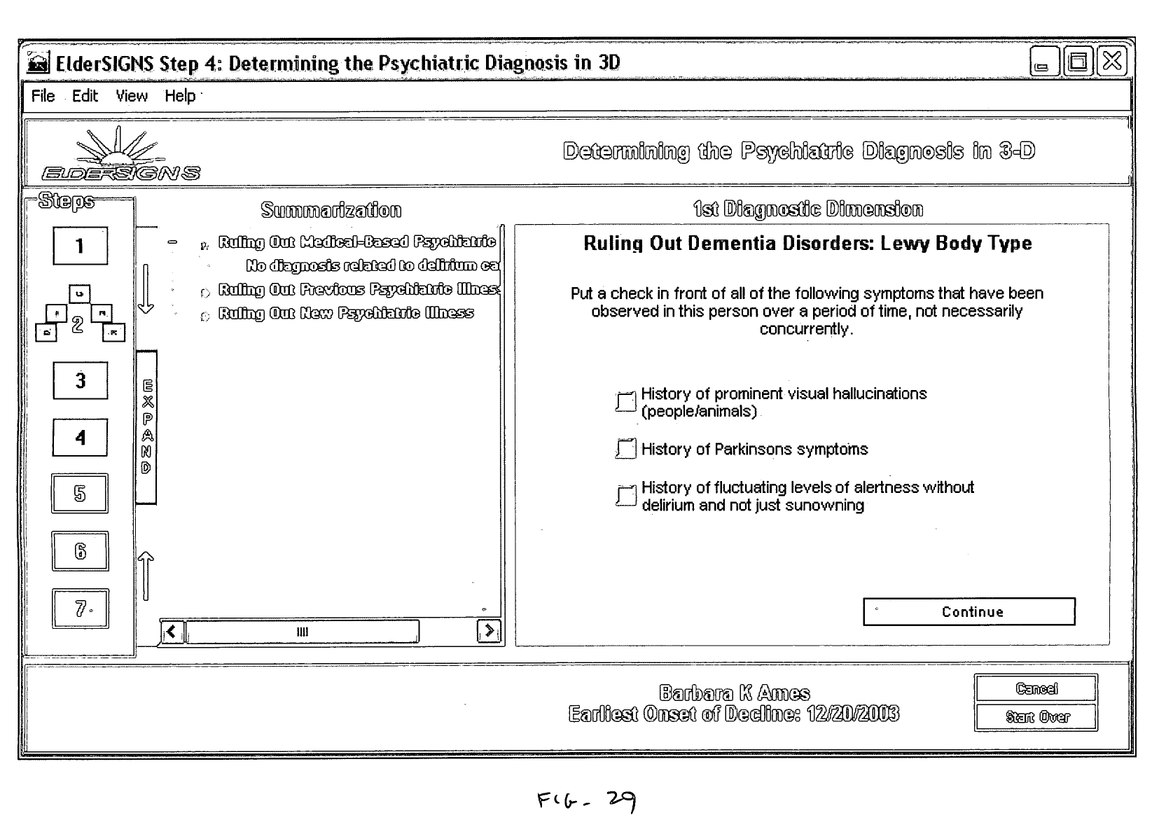

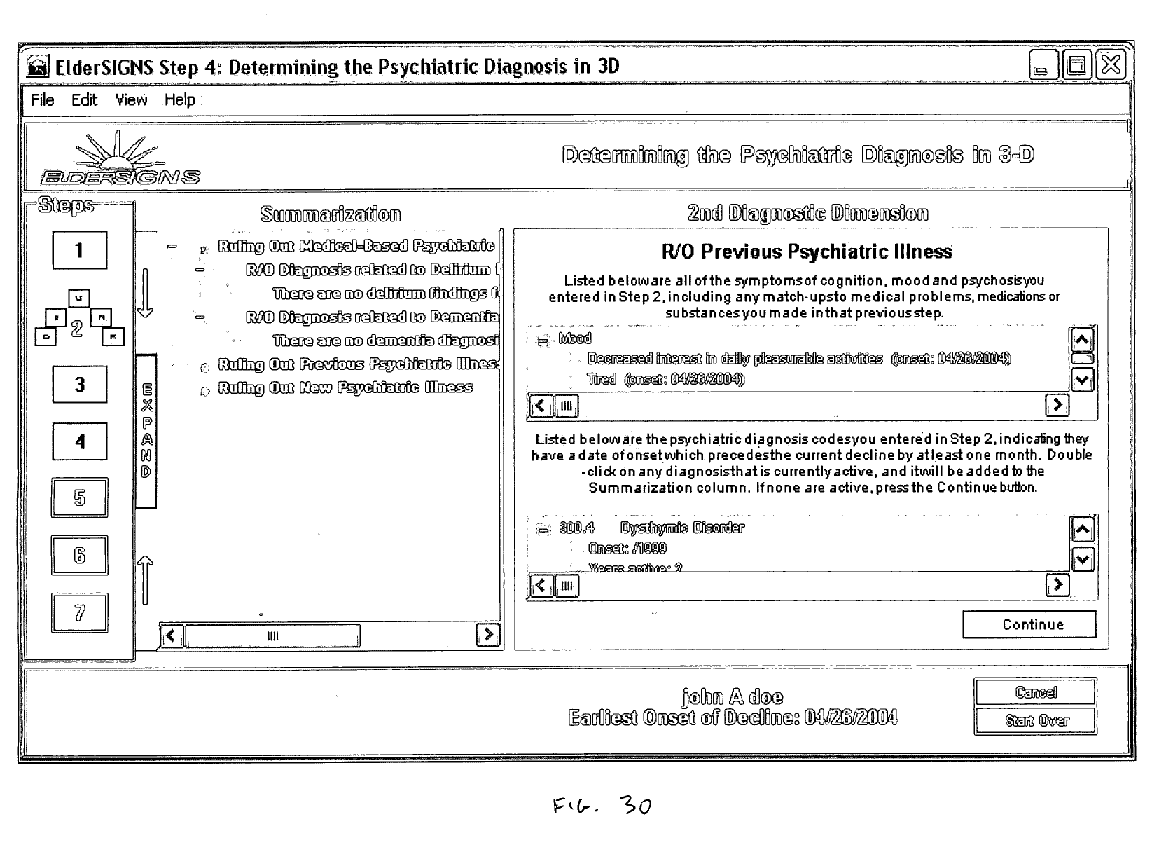

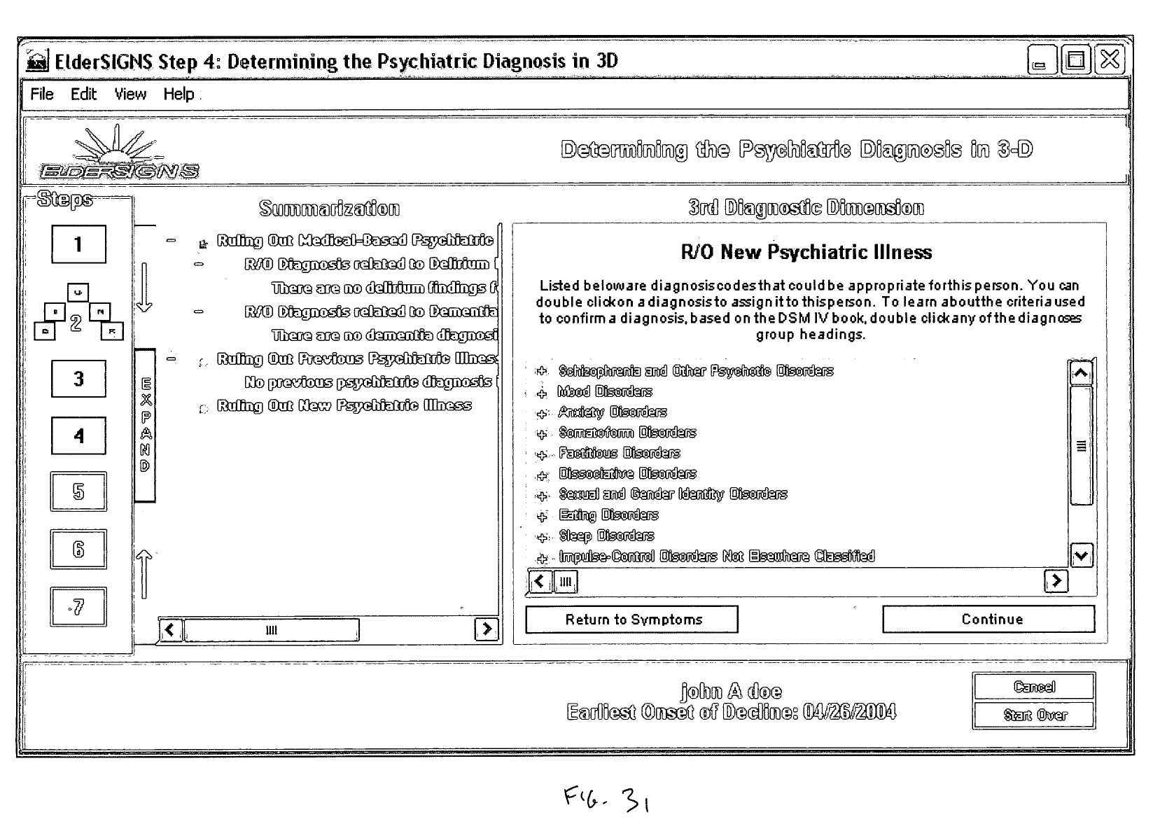

As shown in FIGS. 27-31, the software structures the user to rule-out out psychiatric disorders in a three dimensional fashion. In the first diagnostic dimension, the user is structured to rule out delirium and dementia processes as well as other medically related psychiatric disorders. The software will automatically give the proper DSM-IV diagnosis of delirium and dementia as well as its specific type based on the clinical data inputted by the user. The software will also prompt the user for a dementia workup status and any holes in that work-up will automatically prompt the user to finish the complete work up on Step 5. If a medically related psychiatric diagnosis is given of dementia and/or delirium, dimension 2 and 3 are not available to be opened because no other diagnoses can be given. If there is no diagnosis in dimension 1, then the acute symptoms of psychiatric illness are then potentially matched up to previous psychiatric disorders on dimension 2, or newly diagnosed psychiatric disorders on dimension 3, using strict DSM-IV criteria. Any new psychiatric diagnoses then go automatically to the psychiatric history data base. Any previous psychiatric diagnoses not diagnosed acutely become designated ‘history of . . . ’ in the data base.

In step five, addressing the targeted declines 150, the user is prompted to address the targeted declines and the first four clinical areas before considering psychiatric management of any behavioral problems or functional decline. Furthermore, the user is prompted to address all the identified factors under SIGNS to be contributory to the decline by guiding the user through a care plan for each factor. The program will guide user through a care plan for each factor and will aid the user in developing a simple but effective behavioral plan for any targeted behavioral decline, thereby ensuring a comprehensive, safe, and effective plan is put into effect before even considering psychiatric medication management.

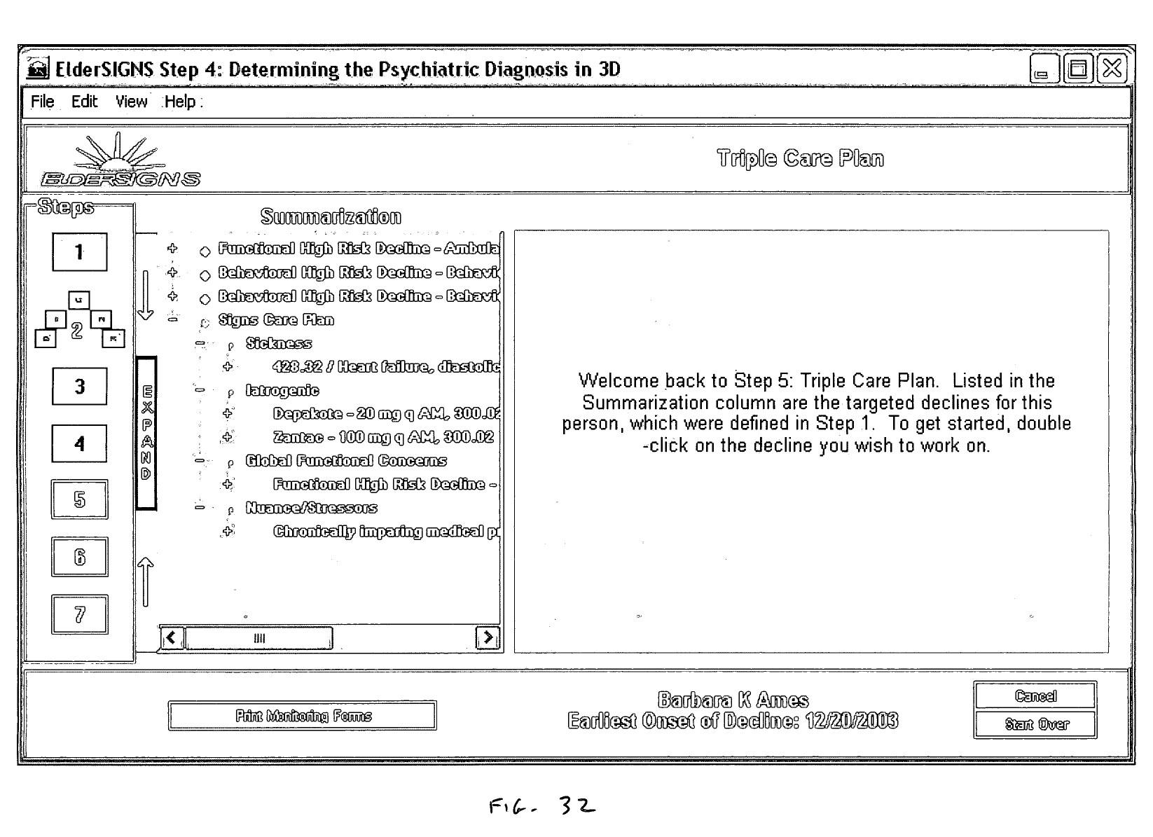

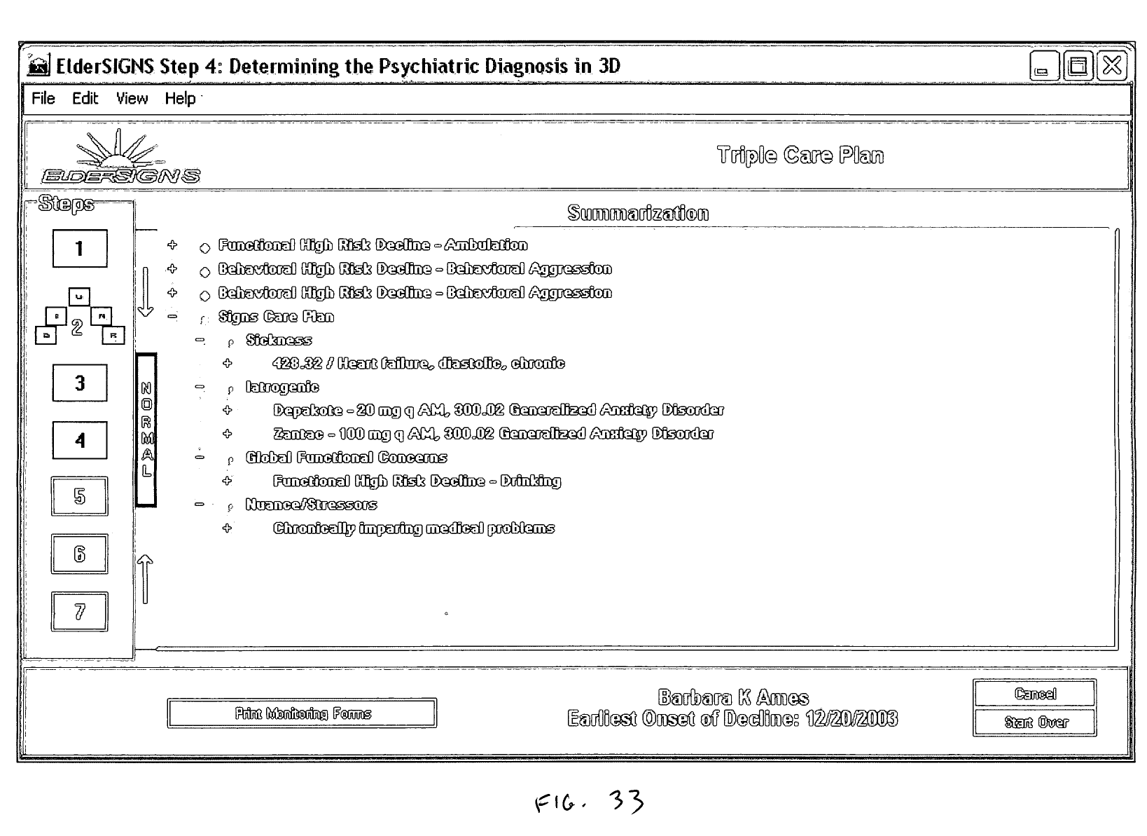

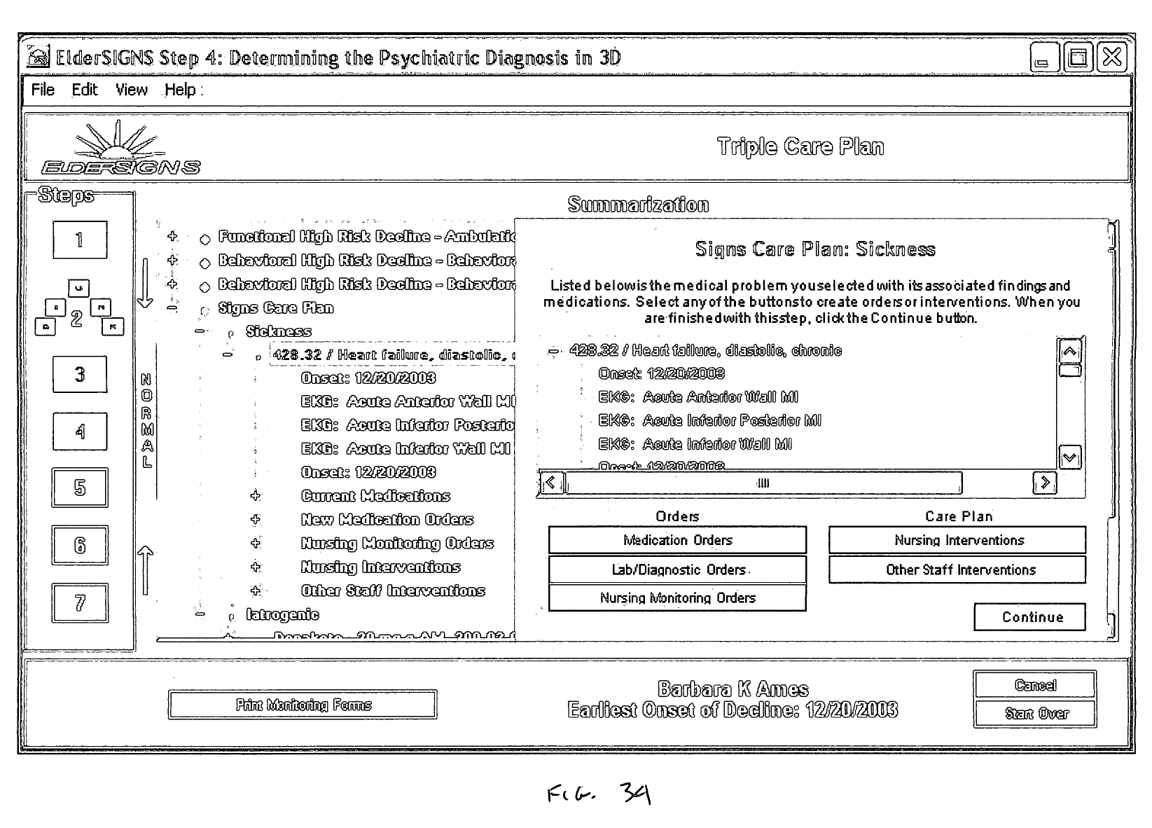

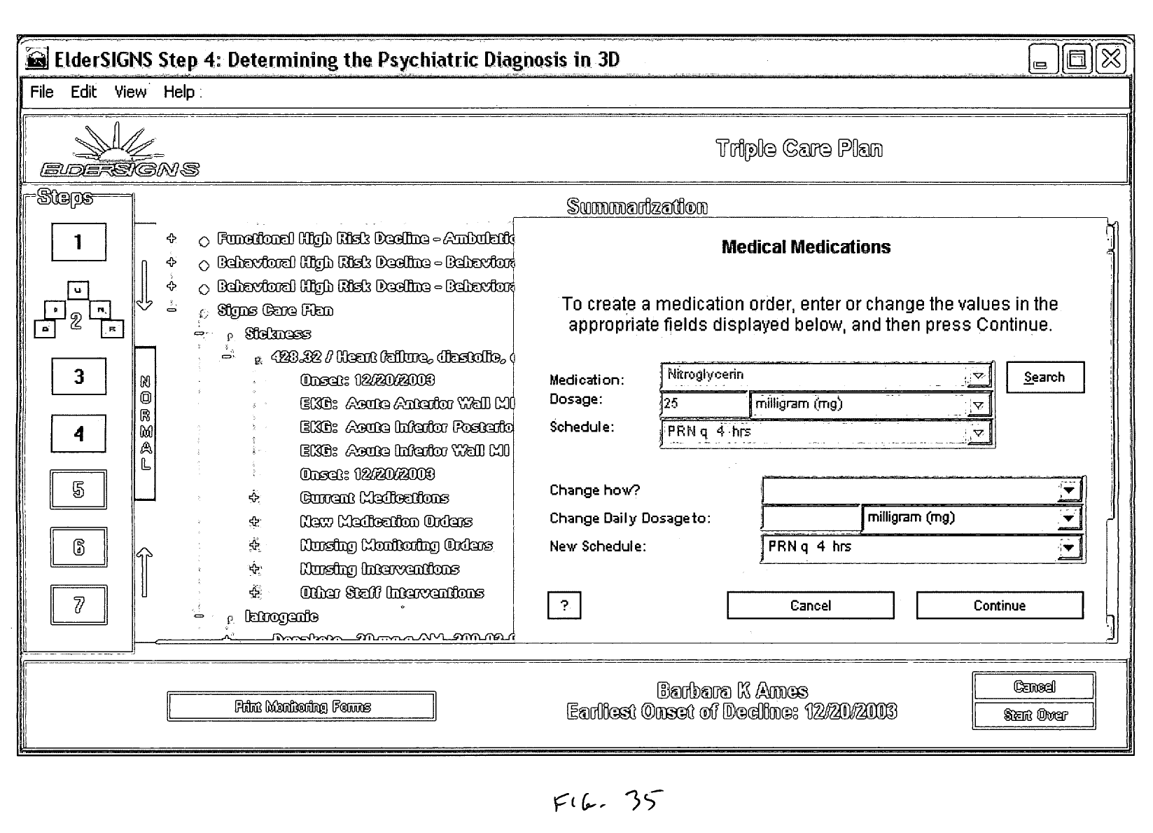

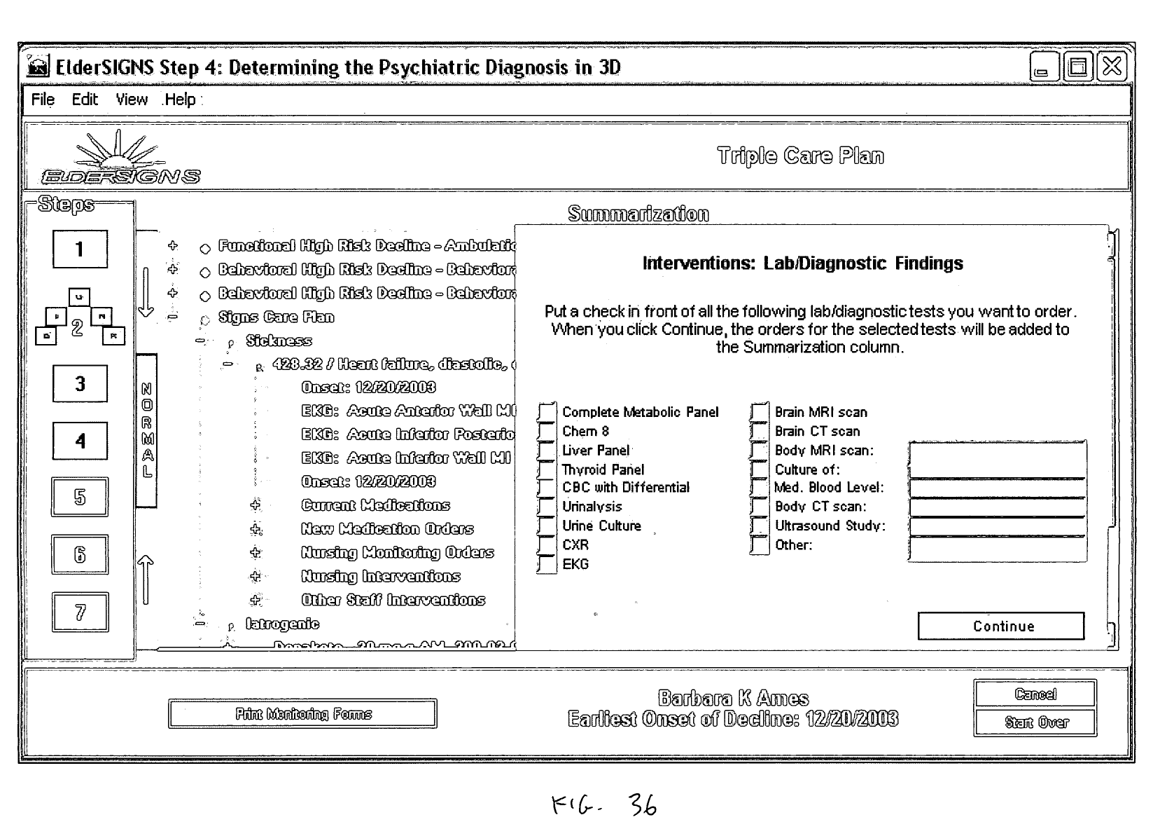

Depicted in FIGS. 32-36, the software takes all of the information of the targeted decline/s from Step 1 and the identified contributory factors from Step 3 and automatically lists them for treatment under Step 5. In regards to the functional and behavioral targeted decline, the user is structured and guided through a comprehensive care plan process including reminders to address important situational triggers related to the decline. This process includes setting up the goal, the objectives if any, the interventions (for all staff and/or specific staff), as well as care plan orders (medication and non-medication orders). Each component of the care plan has the capacity for building library items for the user to choose from or to type in on his own the different aspects of the care plan. All treatment team members have user access to this step to make intervention recommendations.

The third care plan is the SIGNs care plan. All the contributory factors selected in Step 3 show up here for treatment. In this care plan, the user chooses which contributory factor to treat and then can input interventions and orders to address the problem.

Also in step 5 is the development of the monitoring flow sheet for the targeted decline/s that the user wants to formally monitor. The user is prompted to input specific data to complete the monitoring form and then it is printed and ready to use by the designated staff.

Furthermore, the clinical supervisor can edit the care plans and the flow sheet monitoring forms and then when finished makes a determination of how soon to follow-up on the targeted declines and SIGNs factors being treated. during periods of monitoring between followups, all clinical team members can go back into Step 5 to make any recommendations for change in the care plans. Upon the next followup, the clinical supervisor reviews and edits the changes and when complete adds the new care plan changes to the original one.

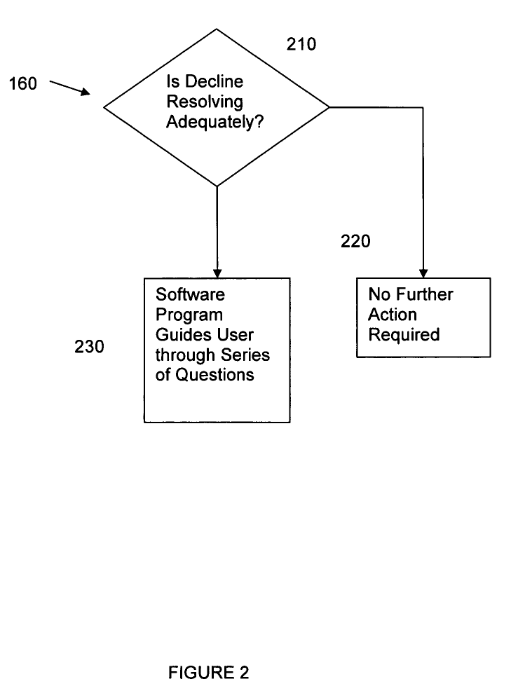

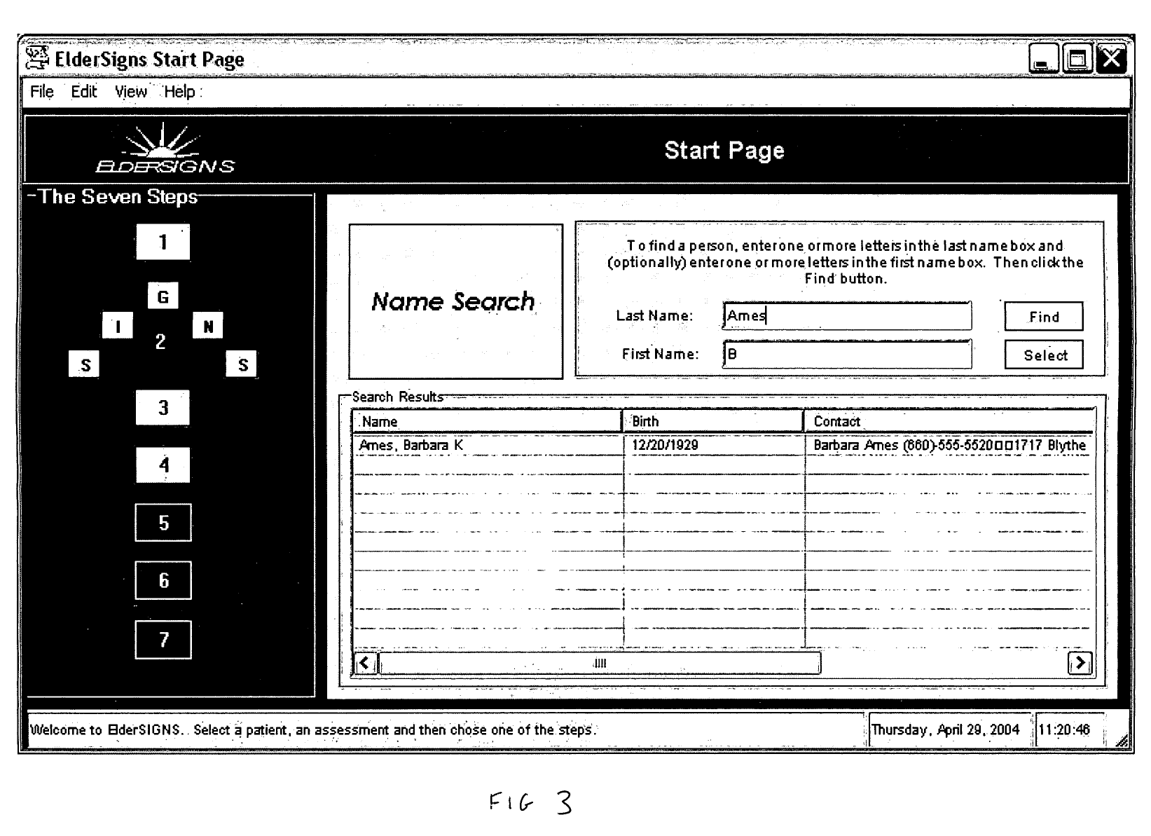

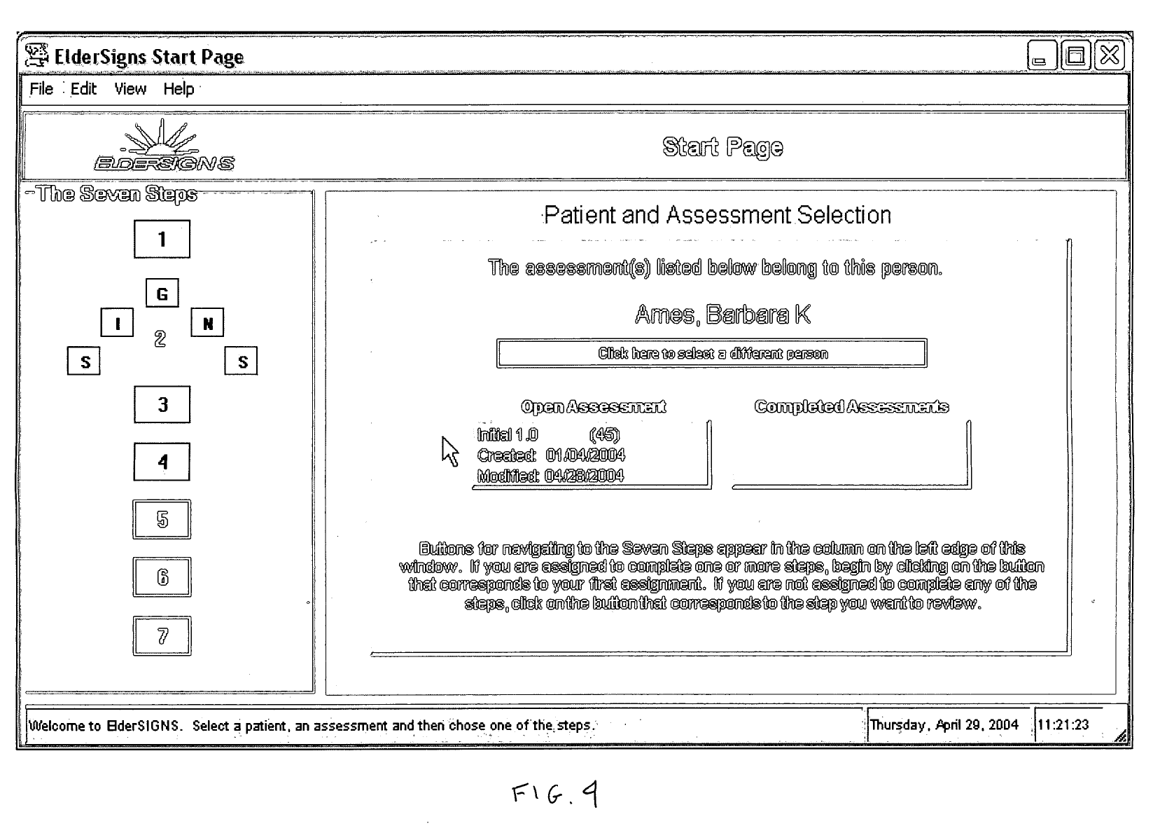

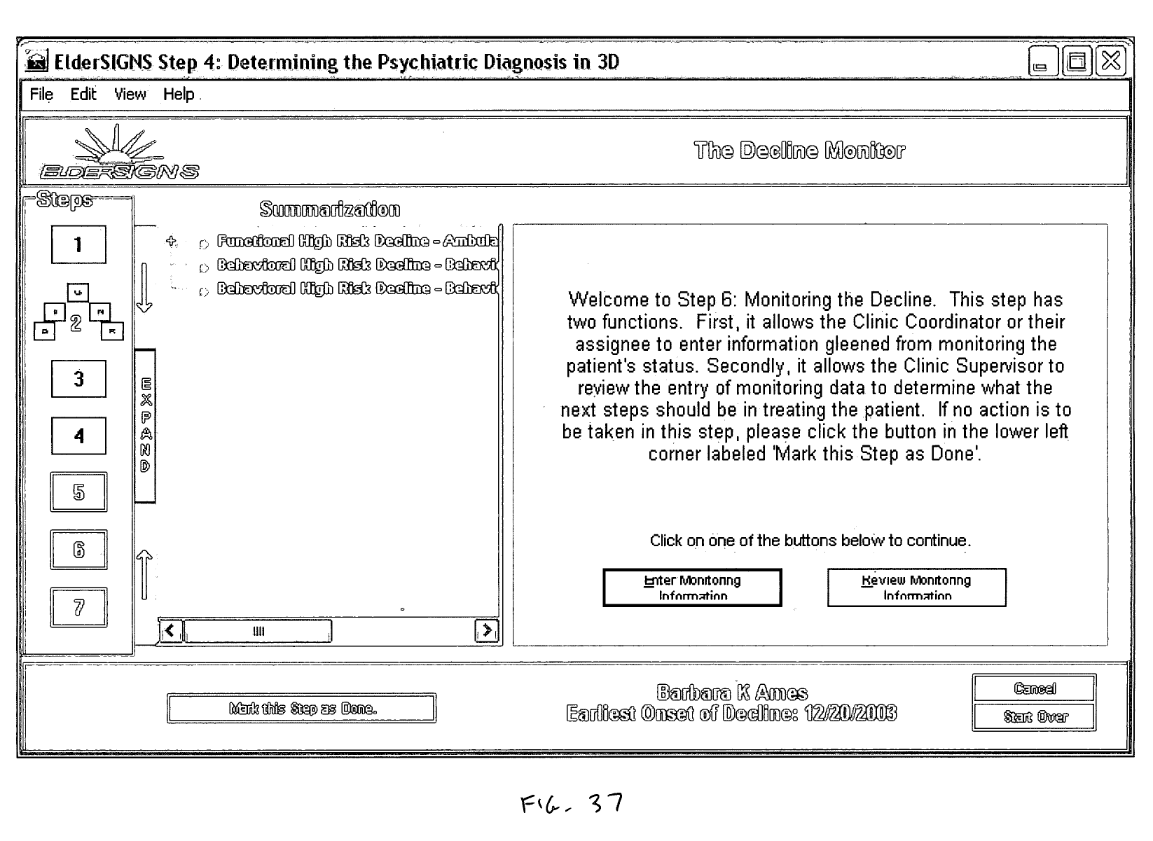

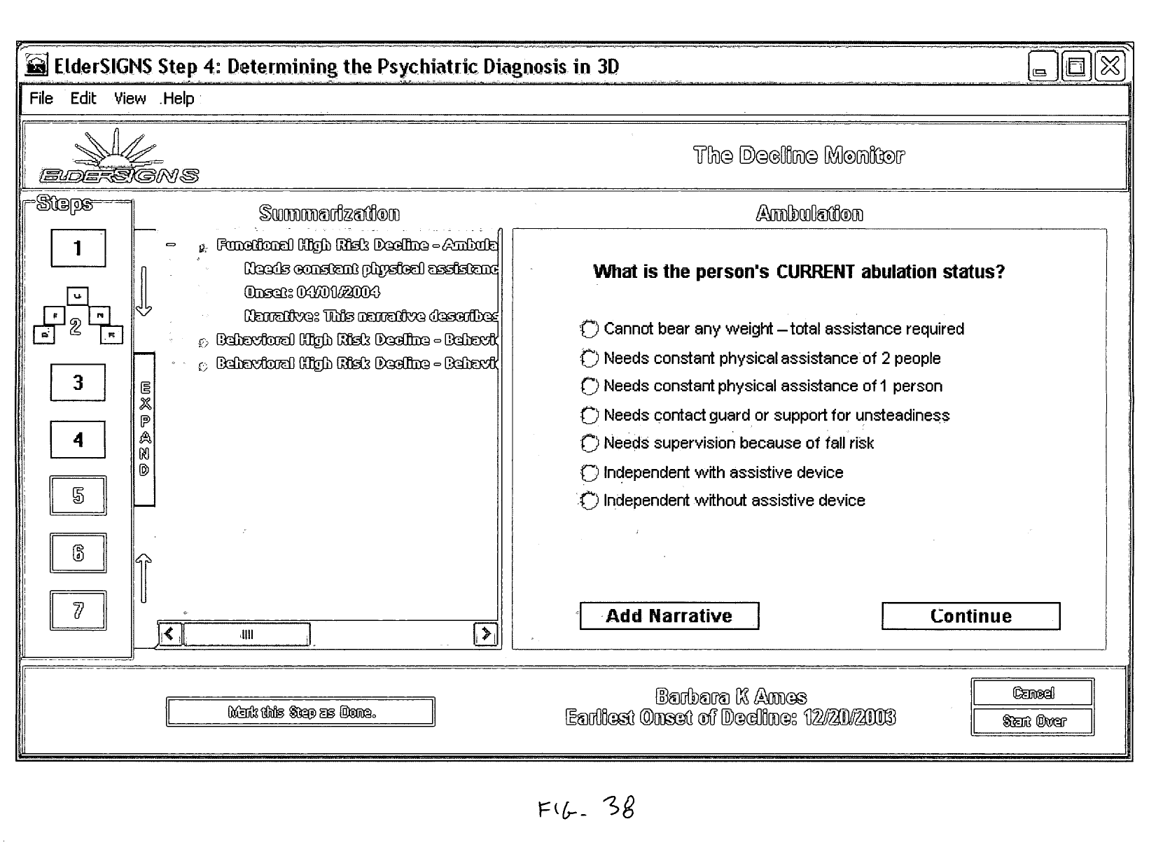

Referring now to FIGS. 1-2, and 37-38, step six is to ensure adequate management of medical and medication factors 160. If the decline is still occurring and not resolving adequately enough through the interventions in step 150, the user will then be guided through a series of questions to determine what type of psychiatric medication is best for the specific psychiatric symptom group that is determined to be driving the decline. In step 210, the software program aids the user in determining if the decline is resolving adequately enough through the interventions in step 150. If it is not, then the user is guided through a series of questions 230 to determine what type of psychiatric medication is best for the specific psychiatric symptom group that is determined to be driving the decline. Otherwise, step 220 is performed requiring no further medical intervention. The designated user inputs the data collected from the monitoring flowsheets. The clinical supervisor then evaluates the monitoring data as well as other clinical data and makes the determination of what to do next. Options are to close out the assessment, to close out specific care plans, or to keep all care plans going and to set up the next time for follow-up.

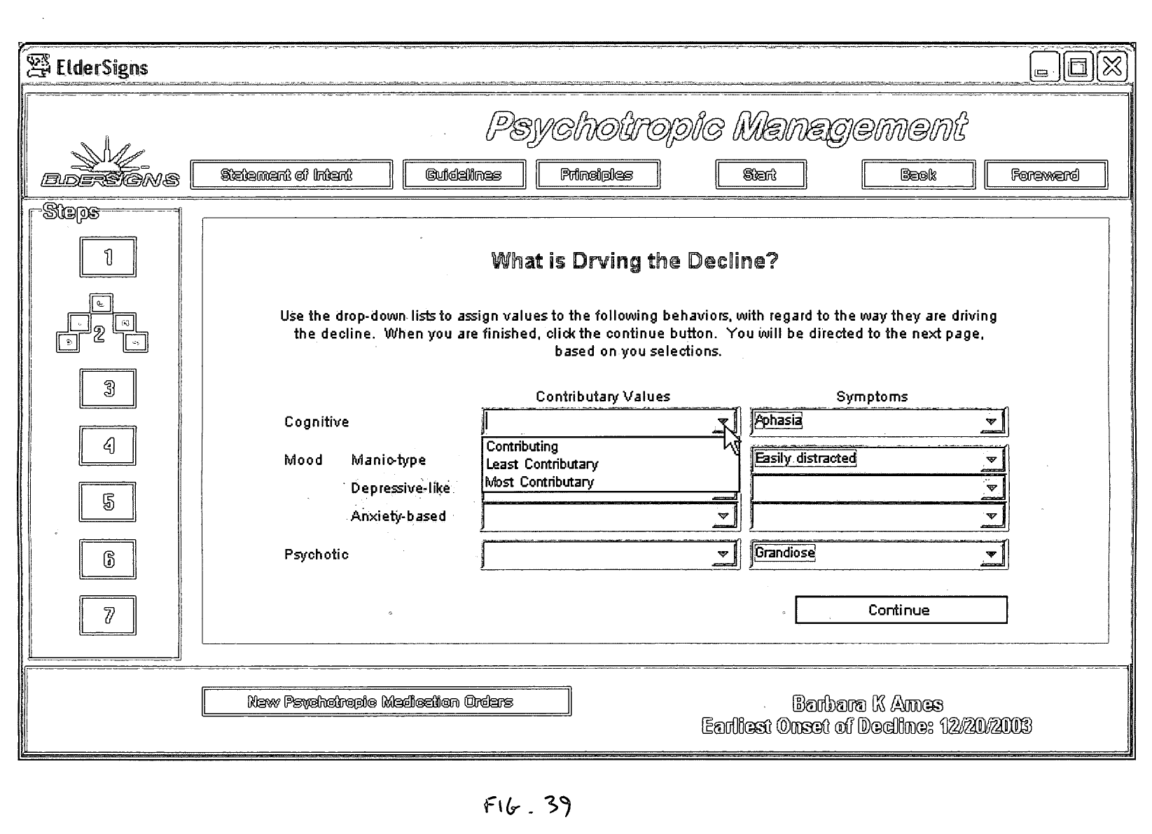

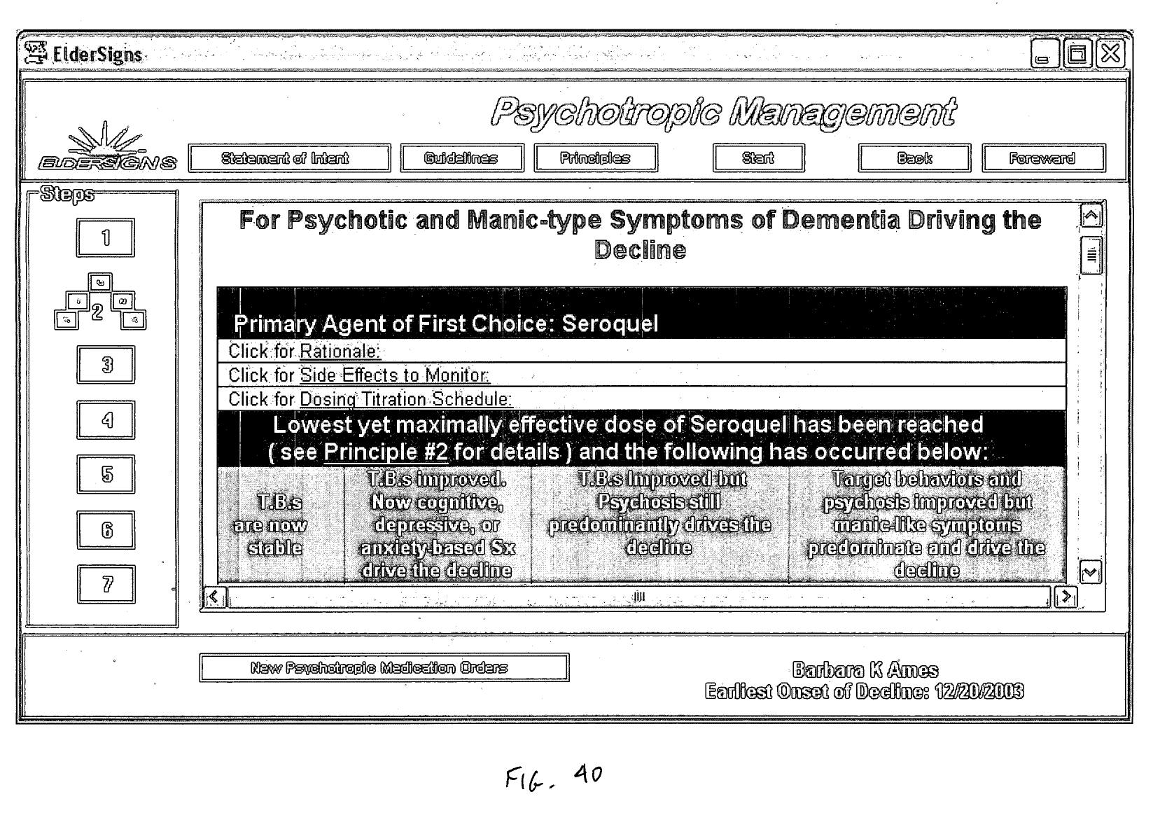

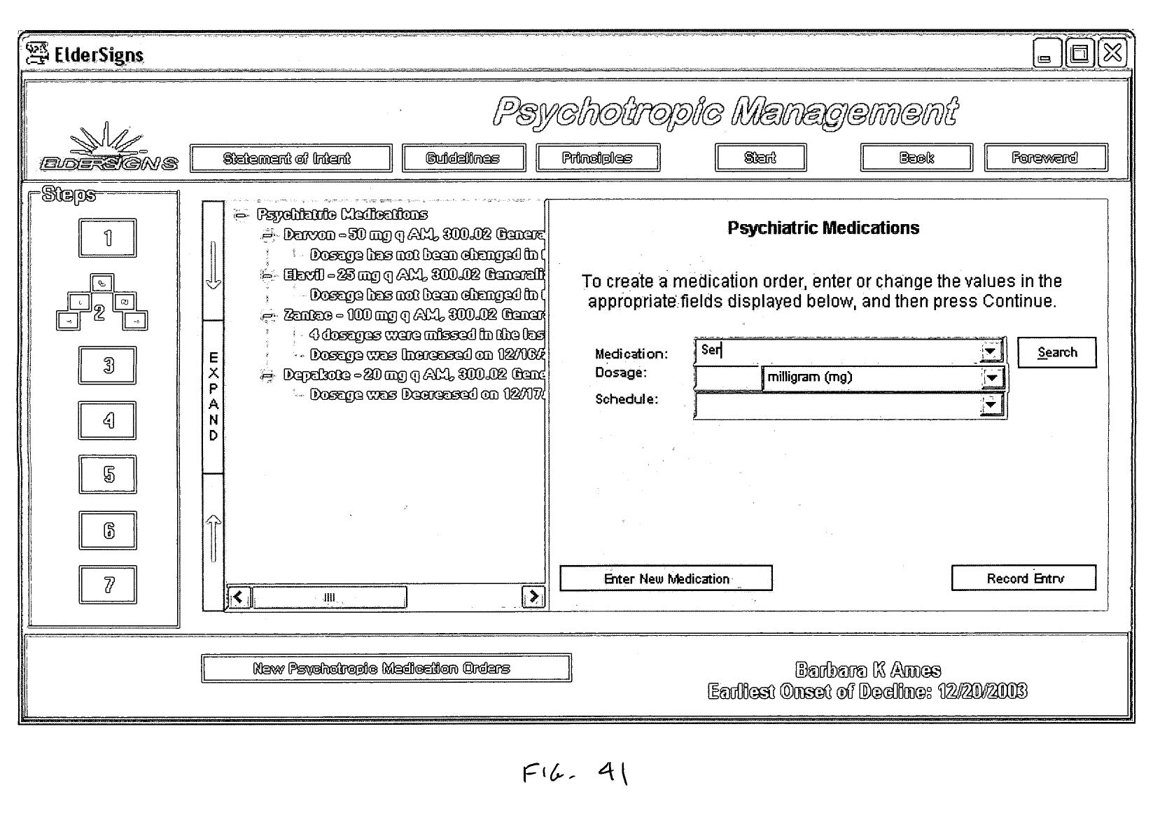

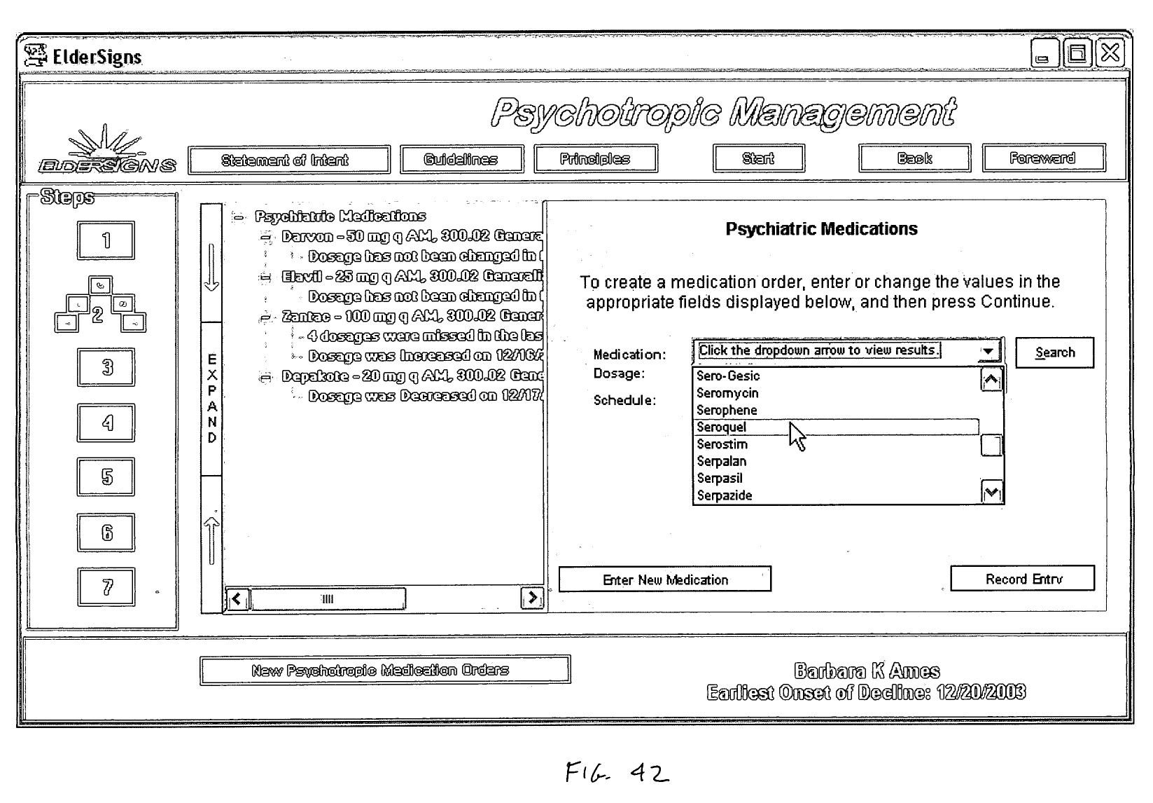

Step seven is directed at psychotropic management 170, depicted further in screenshots in FIGS. 39-42. The user has the option of using the psychotropic algorithm for the treatment of behavioral problems in the elderly related to dementia. When using the algorithm, the user is first prompted to decide which psychiatric symptom group is driving the behavioral decline. Once that is decided, the software program automatically takes the user to the appropriate part of the psychotropic management algorithm for help on determining the safest and most effective psychotropic.

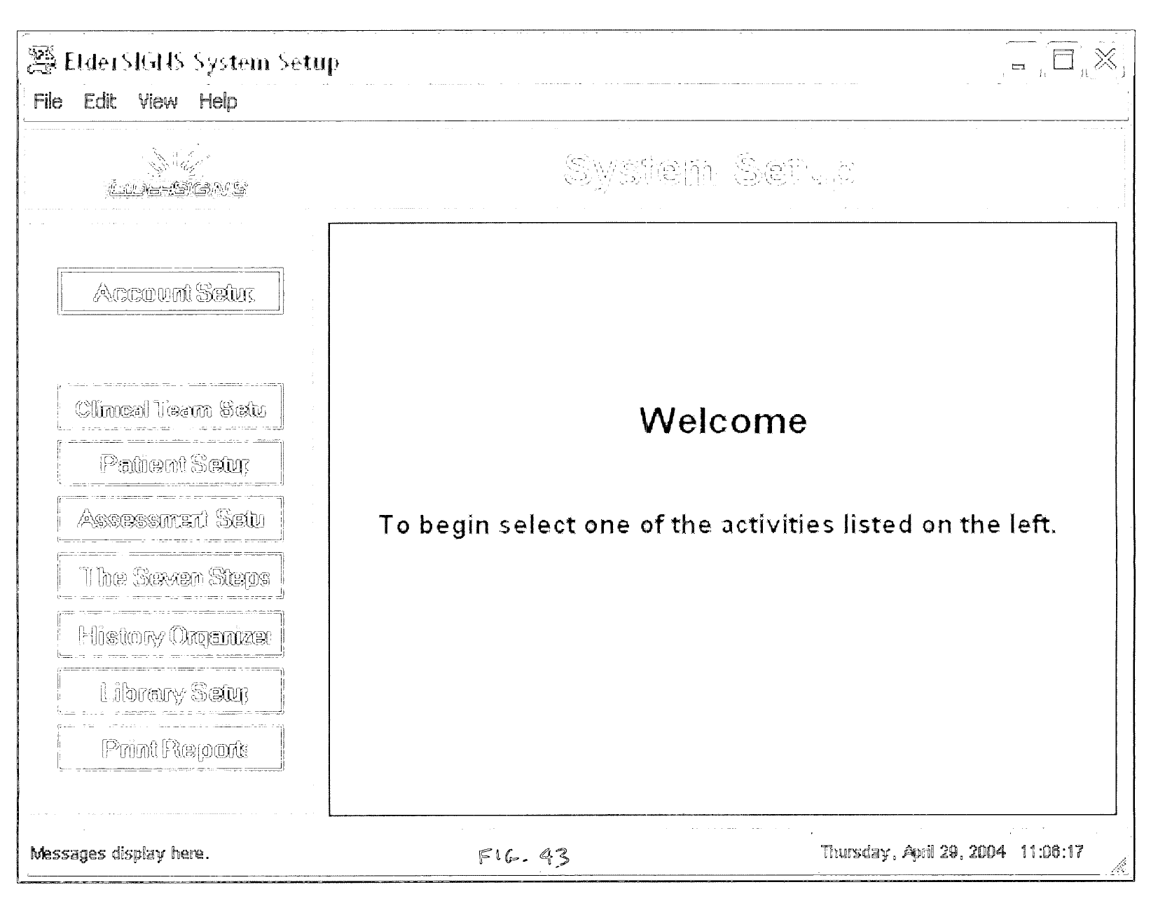

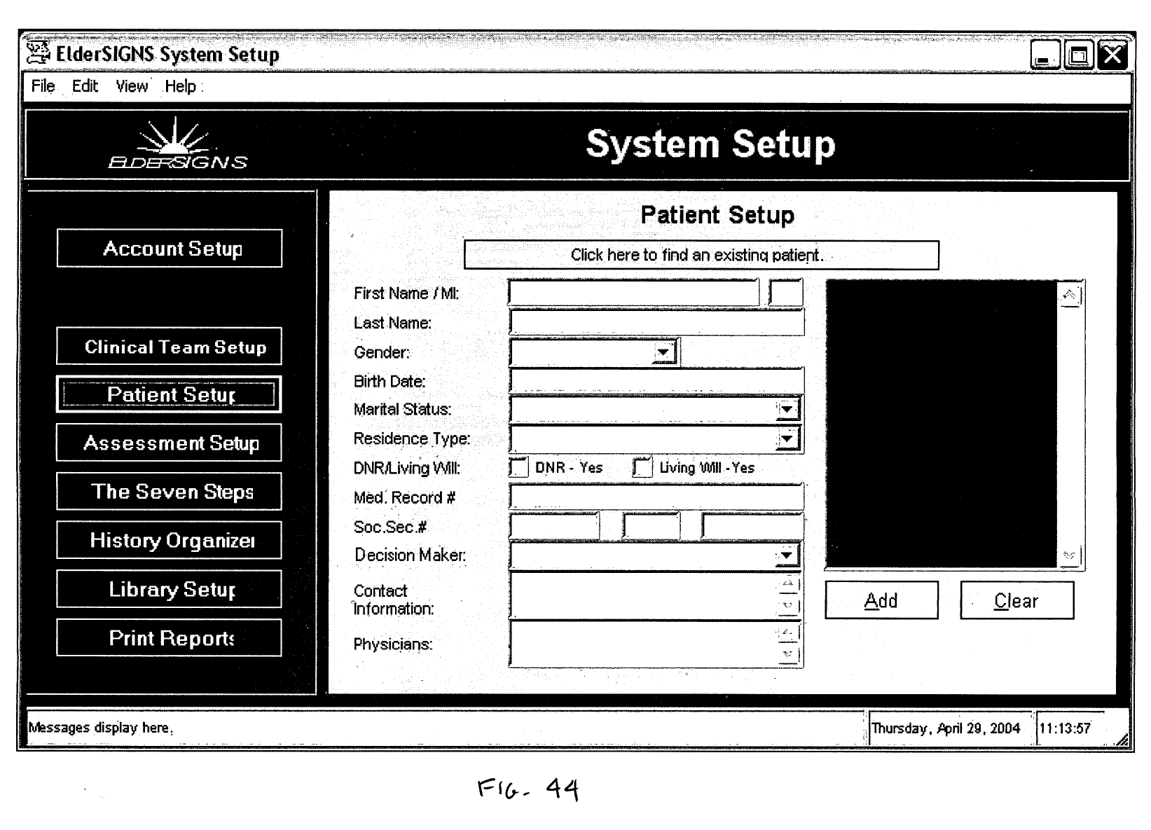

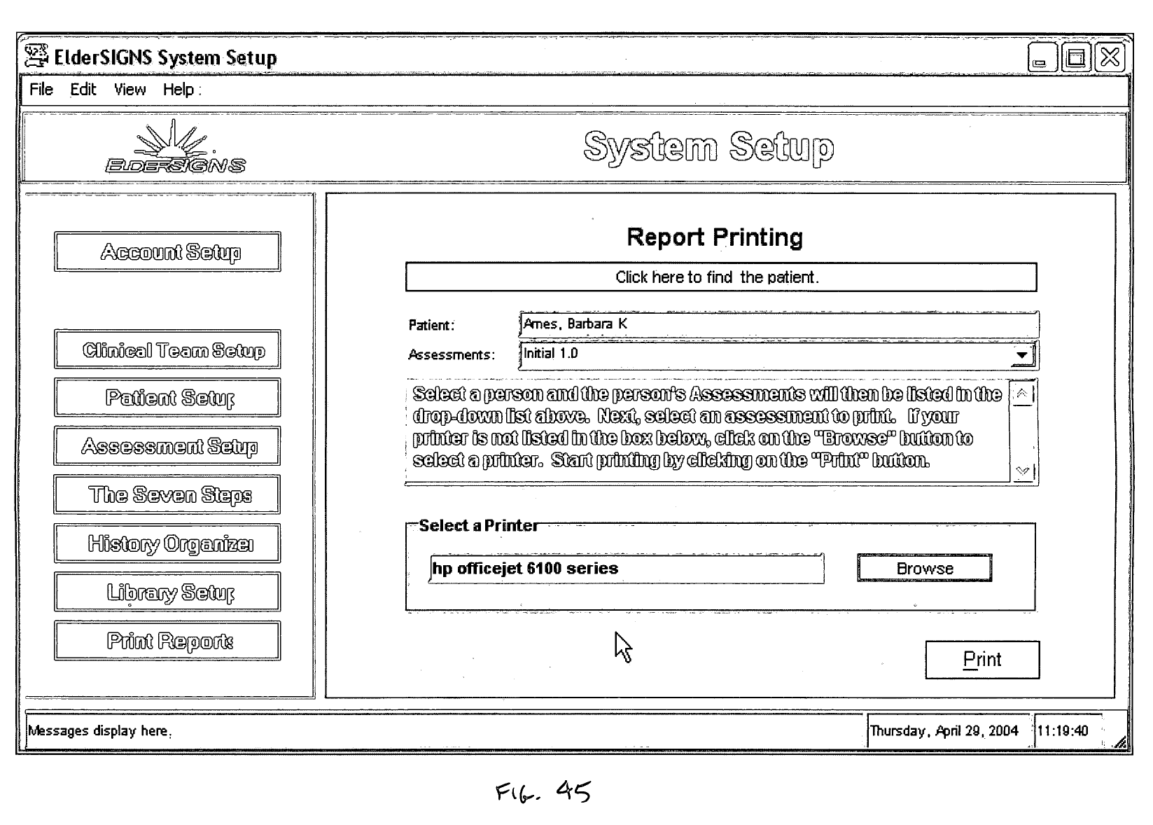

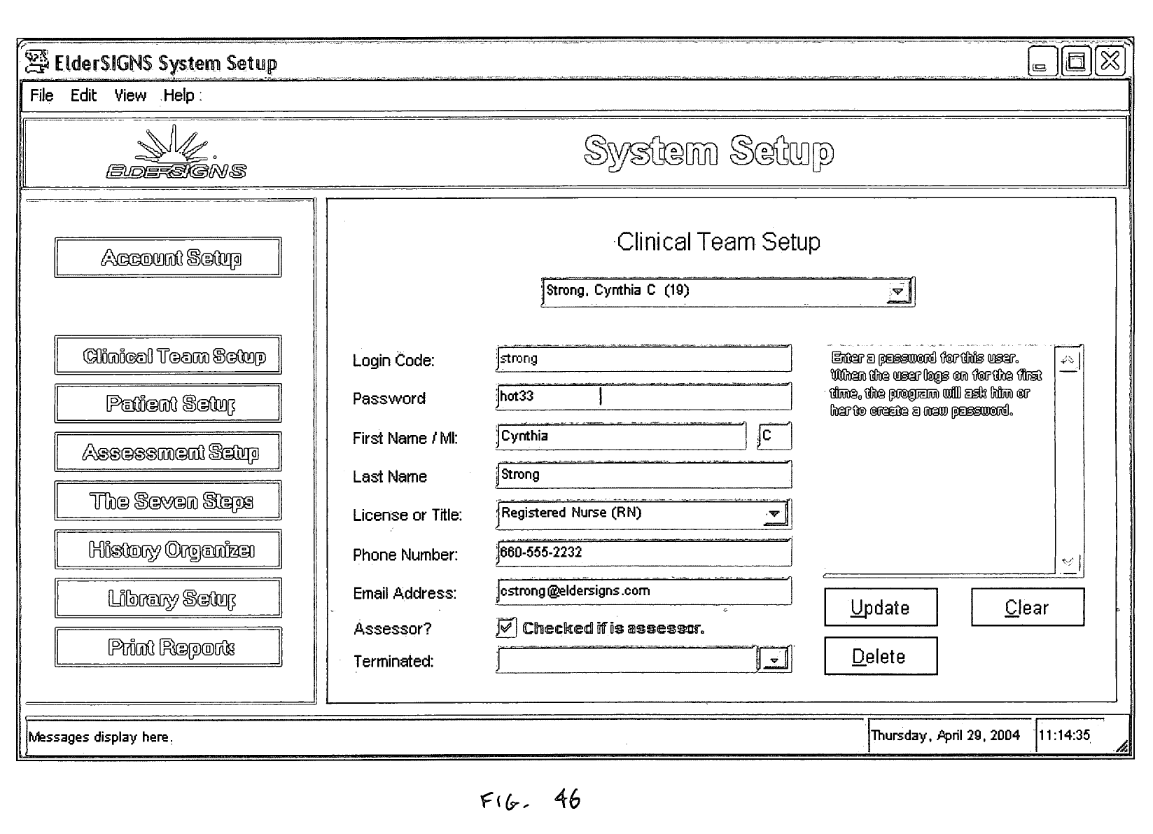

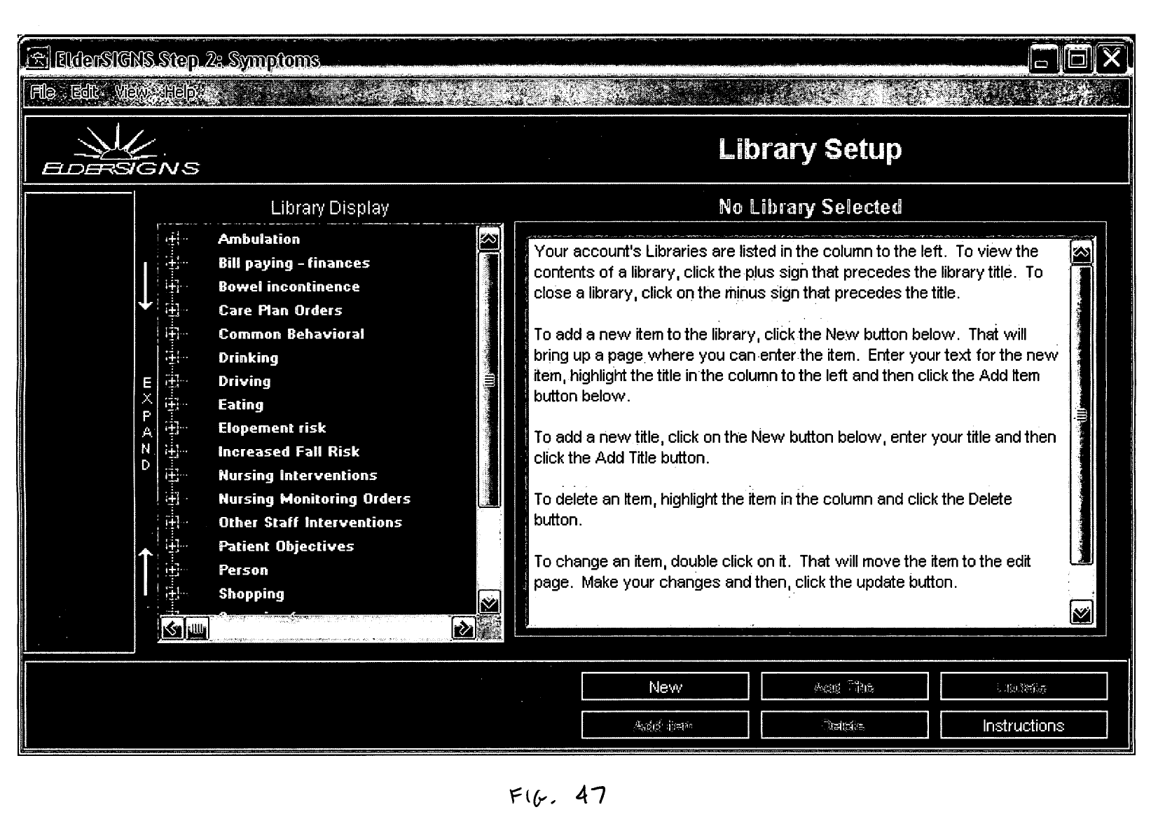

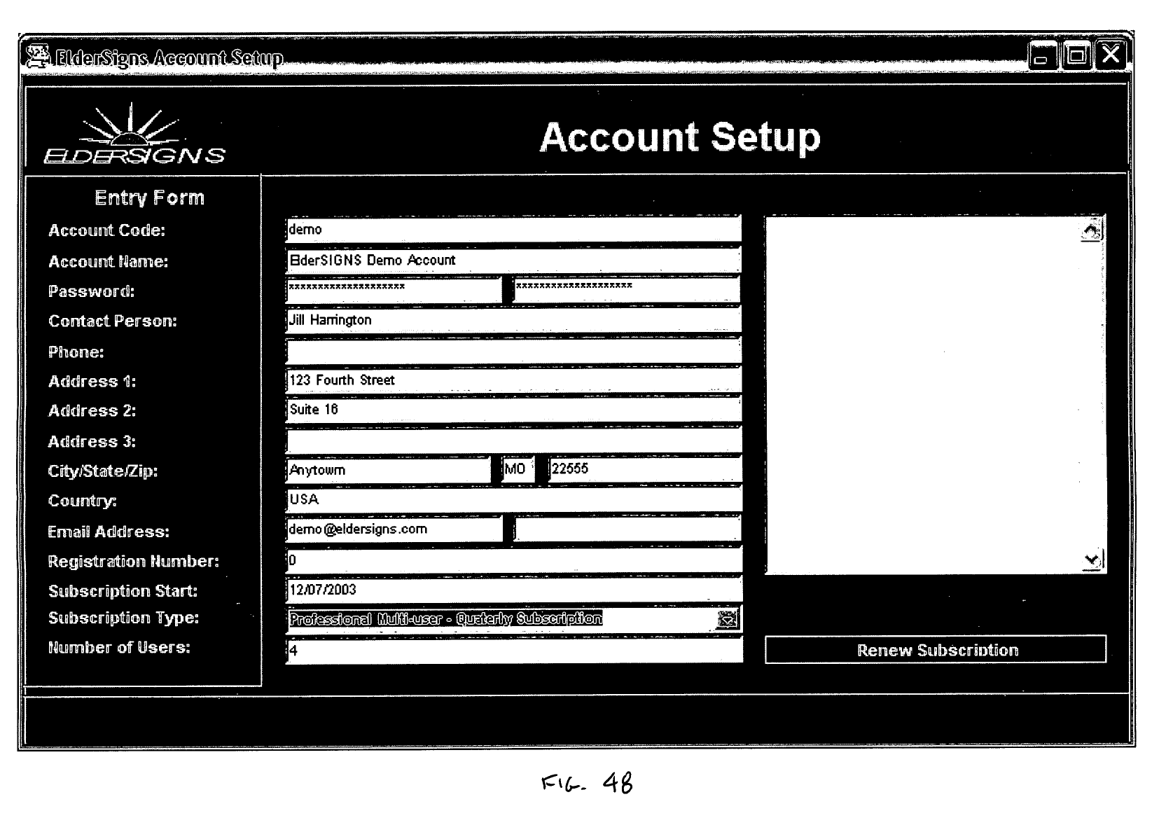

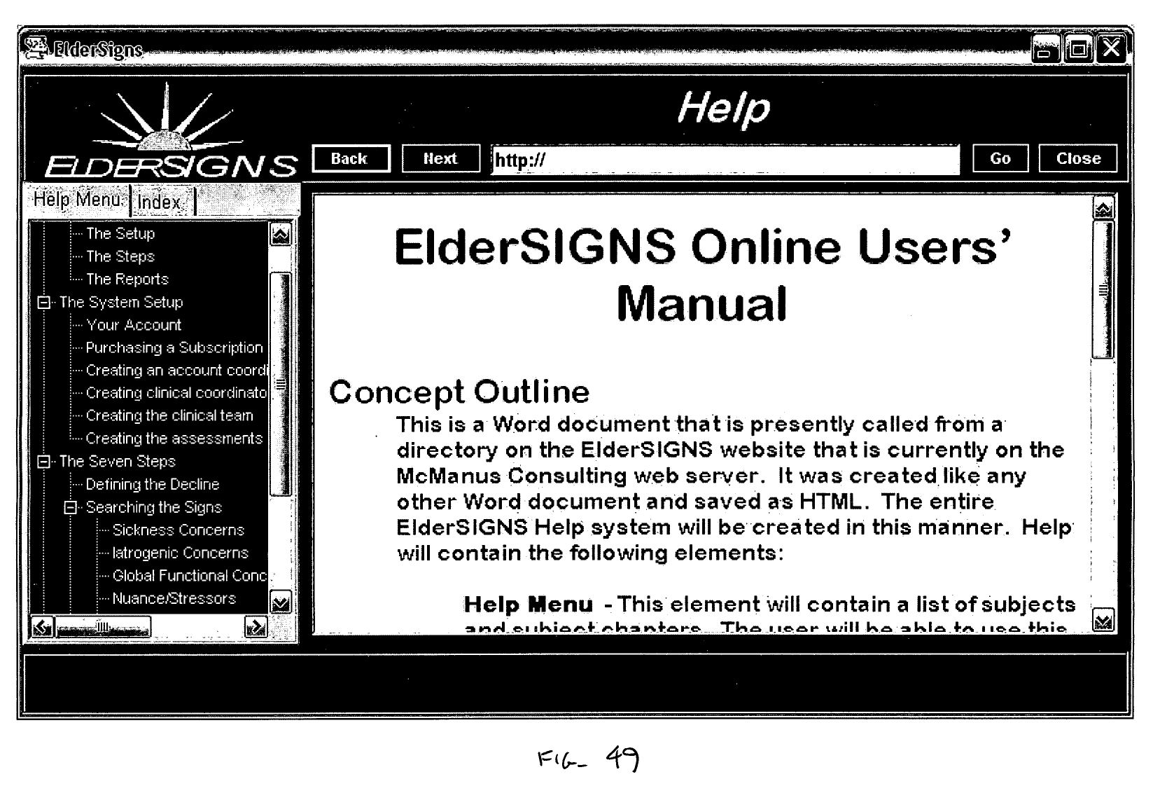

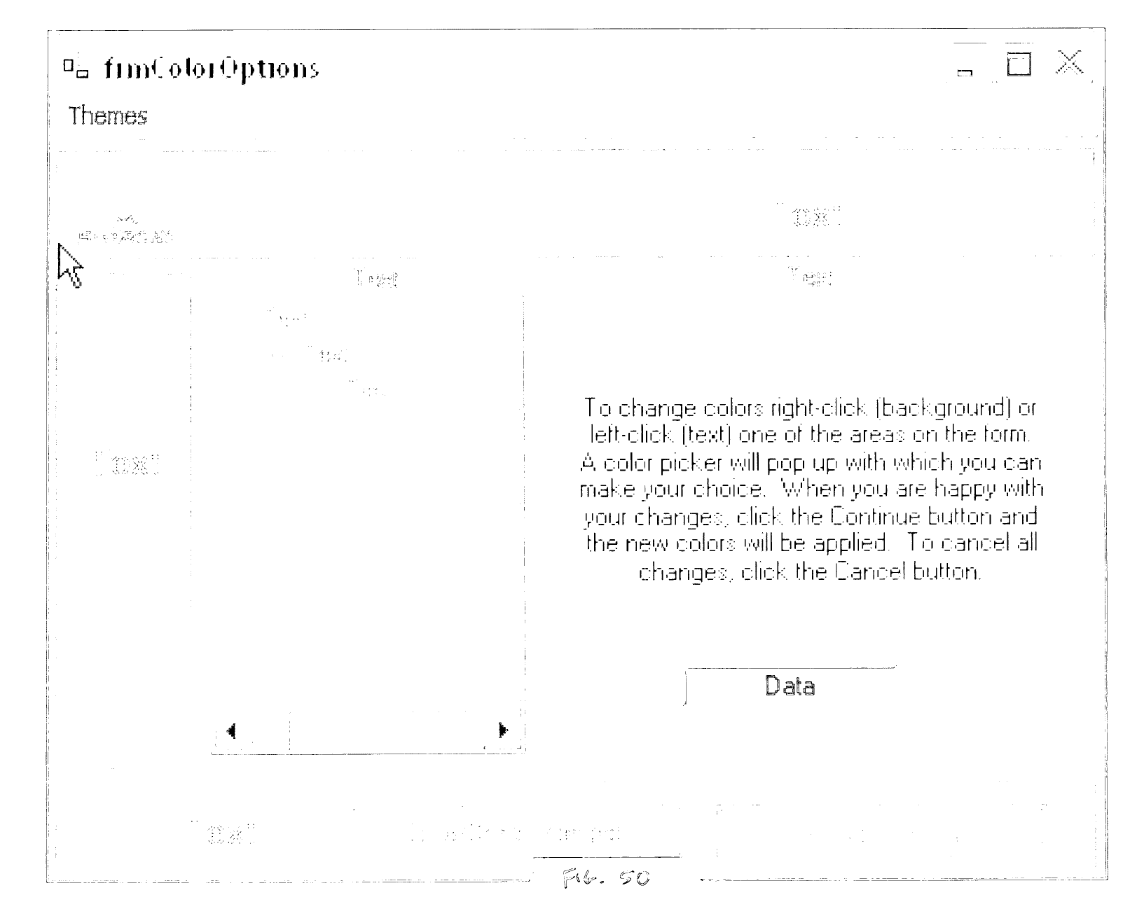

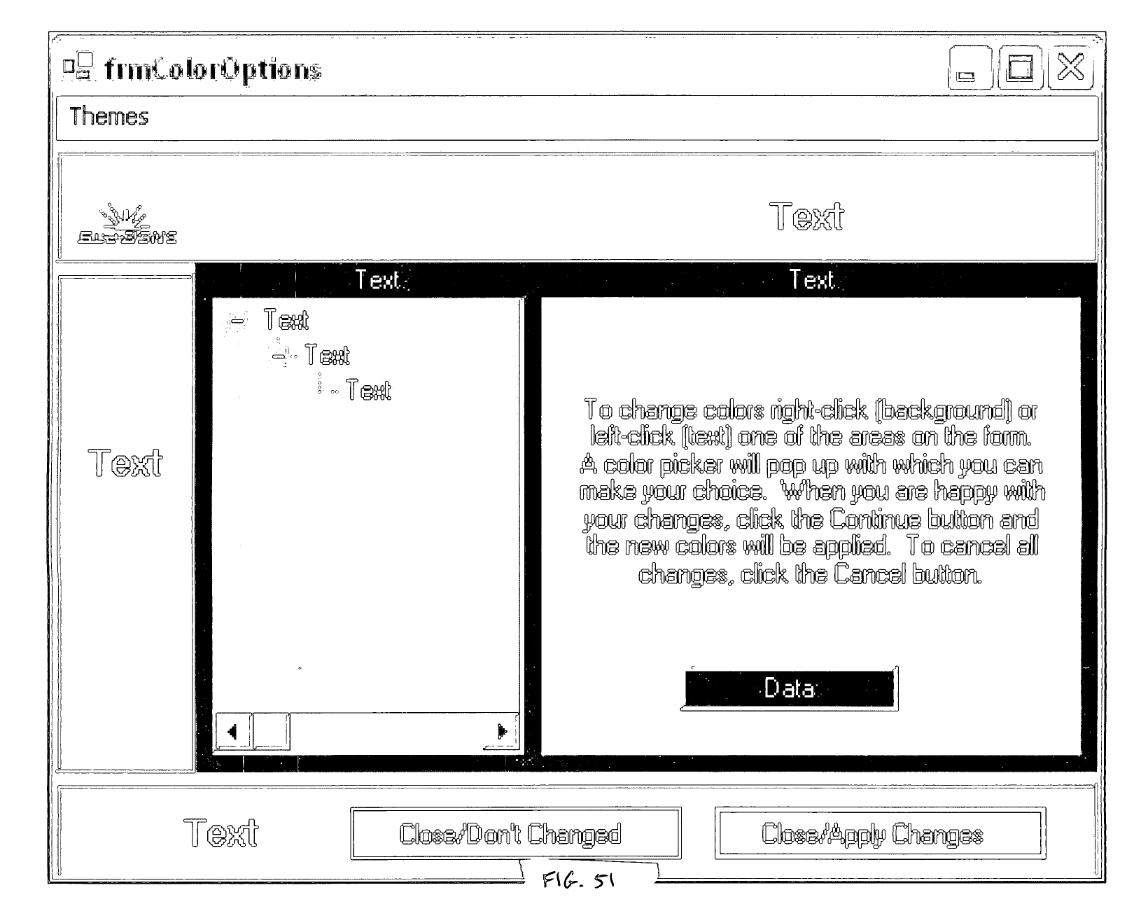

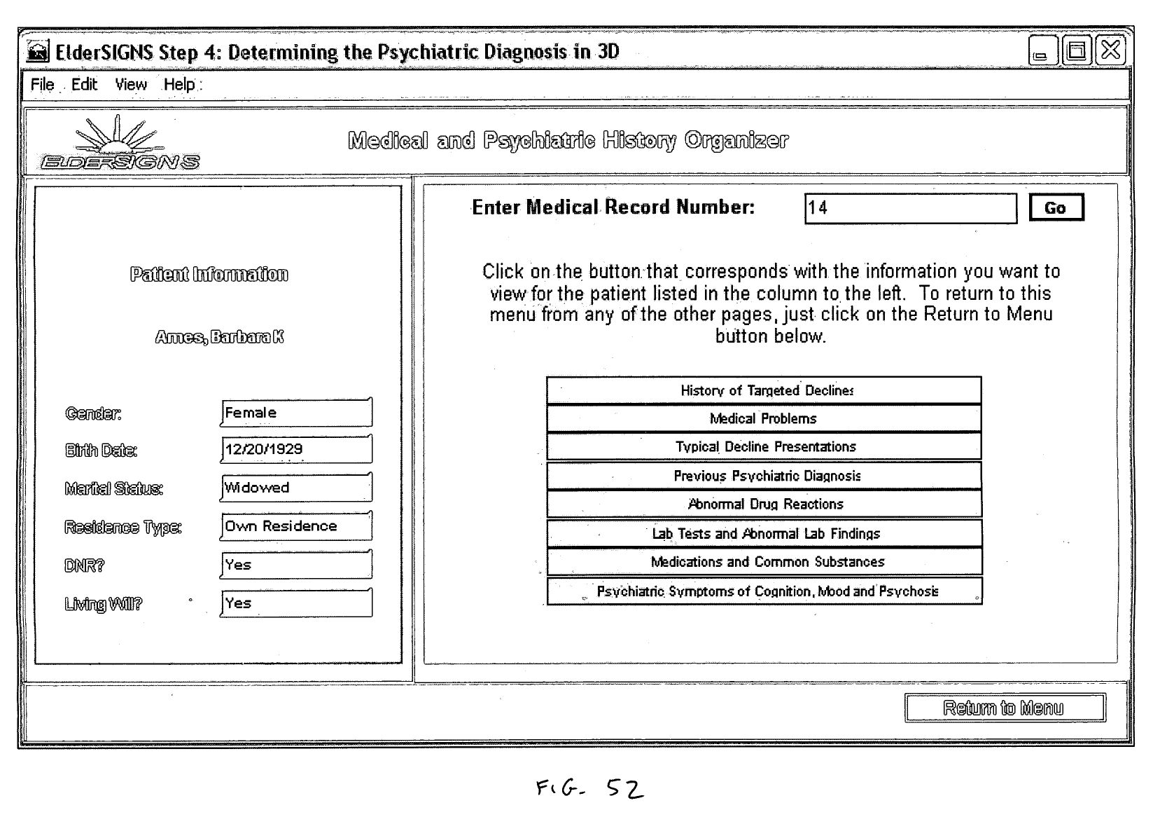

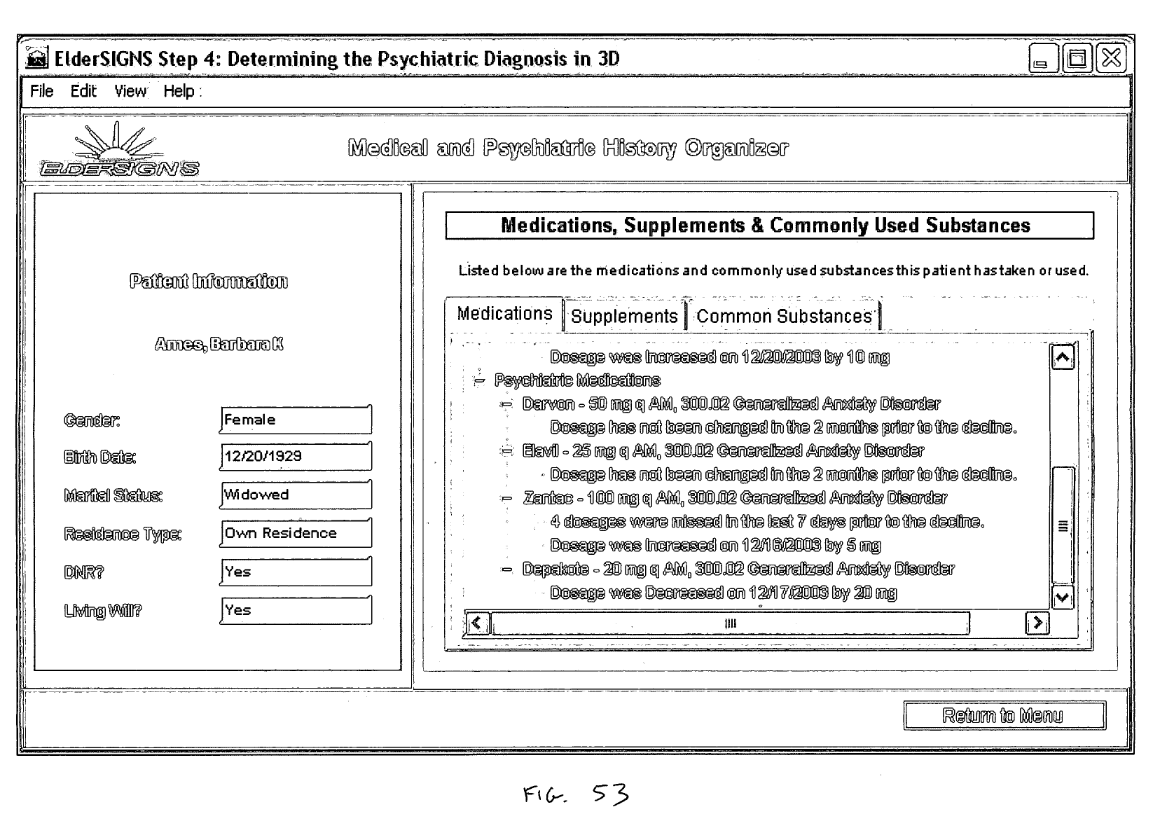

FIGS. 43-53 depict various setup screens for the software program. From these screens, the user can setup a patient's file, make account settings, and setup the format and look of the windows, among other typical settings.

Once the user makes a decision which psychotropic to start/stop/or change, he can then input the medication along with its dosage and schedule.

In a preferred embodiment of the present invention, the software program would be developed to differentiate between single users and facilities that have multiple users. Importantly, enhanced security features can be incorporated into the multiuser version of the software program to prevent users who are not authorized to view certain steps and results from seeing confidential patient information. For example, in a preferred embodiment of the present invention, a Health Insurance Portability and Accountability Act (HIPAA) compliant assignment process in which a designated clinical coordinator can assign certain staff to do certain steps or portions of steps may be employed. In this manner, only staff assigned to that step or portion of the step may access, add, or edit information to that step. However, any known security means known to one of ordinary skill in the art may be employed to protect client confidentiality.

In view of the foregoing disclosure, some advantages of the present invention can be seen. For example, a novel software method of determining and treating psychiatric disorders has been described. Unlike other assessment programs on the market, this novel software method guides a user through from beginning to end in regards to the assessment, diagnosis, treatment and monitoring of acute functional and behavioral problems, particularly of elderly patients.

While the preferred embodiment of the present invention has been described and illustrated, modifications may be made by one of ordinary skill in the art without departing from the scope and spirit of the invention as defined in the appended claims. For example, sickness, iatrogenic concerns, global functional concerns, nuance-stressors, and symptoms of psychiatric illness have been described as potential contributing factors. However, any factors known to one of ordinary skill in the art may be included in the list of potential contributing factors.

What is claimed is:

1. A software method of determining and treating psychiatric disorders by prompting a user, comprising the steps of:
defining the decline;
assessing potential contributing factors leading to the decline;
connecting the potential contributing factors to the decline;
diagnosing the decline;
addressing the decline and identifying potential contributing factors, and developing a care plan;
ensuring adequate management of medical and medication factors; and
addressing psychotropic management.

2. The software method of claim 1, wherein the step of defining the decline includes the software prompting the user to define each decline through a series of prompts describing each identified targeted decline in detail using observable and measurable descriptors related to time, situation/place and people involved in the decline.

3. The software method of claim 1, wherein the step of assessing the potential contributing factors includes the steps of assessing sickness, iatrogenic concerns, global functional concerns, nuance-stressors, and symptoms of psychiatric illness.

4. The software method of claim 3, wherein the step of assessing sickness includes completing a full medical and psychiatry history including medical and psychiatric diagnoses along with their ICD-9 codes, identifying certain chronic medical problems that present with repeated behavioral and/or functional decline, a detailed adverse drug reaction summary delineating allergies, medication-specific side effects, and cumulative side effects involving more than 1 medication at a time.

5. The software method of claim 4, wherein the step of assessing sickness further comprises the steps of ruling out important acute clinical problems such as delirium, common physical findings, and extrapyramidal symptoms; and identifying all abnormal lab and diagnostic findings.

6. The software method of claim 5, wherein the step of assessing sickness further comprises the step of prompting the user prompted to match-up the identified acute medical findings with any of the person's chronic medical problems.

7. The software method of claim 5, wherein the step of assessing sickness further comprises the step of prompting the user prompted to match-up the identified acute medical findings with any acute temporary medical conditions.

8. The software method of claim 3, wherein the step of assessing the iatrogenic concerns further comprises the step of putting in all of the patient's medications being taken before the onset of the decline including ones started or stopped.

9. The software method of claim 8, further comprising the step of the software automatically cross-referencing the entire medication list to certain medication concern rule outs that have been triggered.

10. The software method of claim 9, further comprising the step of the software presenting all of the potentially identified medication risk factors in a structured fashion to help the user to determine if the identified medication risk factors are connected to the acute physical findings identified in the assessing sickness step.

11. The software method of claim 3, wherein the step of addressing global functional concerns includes prompting the user to describe any other functional declines not targeted in the step of defining the decline so that they can be identified and monitored.

12. The software method of claim 3, wherein the step of addressing the nuance stressors includes the step of assessing all the potential environmental factors that could be impacting on the patient's targeted declines.

13. The software method of claim 12, wherein the step of assessing all the potential environmental factors that could be impacting on the patient's targeted declines further comprises the step of prompting the user to consider a broad range of environmental stressors.

14. The software method of claim 13, wherein the environmental stressors can be selected from the group including Negative Life Experiences, Physical Discomfort, Previously Tried Decline Interventions, and Previously Tried Preventative Measures of the Decline.

15. The software method of claim 14, further comprising the step of the software prompting the user to identify whether Previously Tried Decline Interventions, and Previously Tried Preventative Measures of the Decline alleviated or worsened the incidence of the decline.

16. The software method of claim 15, further comprising the step of the software prompting the user to add the identifications of alleviation or worsening of the decline to the care plan.

17. The software method of claim 3, wherein the step of assessing the symptoms of psychiatric illness includes structuring the user to divide the symptoms of psychiatric illness into one of cognitive, mood, or psychotic groups.

18. The software method of claim 17, wherein the cognitive group is subdivided into delirium-like and dementia-like symptoms.

19. The software method of claim 17, wherein the mood group is subdivided into depressive, anxiety-based, and manic-like symptoms.

20. The software method of claim 17, wherein the psychosis group is subdivided into delusions, hallucinations, and thought disorders.

21. The software method of claim 17, further comprising the step of prompting the user to match any of the psychiatric symptoms to the acute medical conditions and medication concerns in the steps of assessing sickness and iatrogenic concerns.

22. The software method of claim 3, wherein the step of connecting the potential contributing factors to the decline includes the software automatically receiving the potential contributing factors identified in the step of assessing the potential contributing factors.

23. The software method of claim 1, wherein the step of diagnosing the decline includes the step of structuring the user to rule out psychiatric disorders in a three dimensional fashion.

24. The software method of claim 23, further comprising the step of the software program structuring the user to rule out delirium, dementia processes, and medically related psychiatric disorders in a first dimension.

25. The software method of claim 24, further comprising the step of the software program prompting the user for a dementia workup status.

26. The software method of claim 25, further comprising the step of matching up previous psychiatric disorders to acute symptoms of psychiatric illness in a second dimension.

27. The software method of claim 26, further comprising the step of matching up newly diagnosed psychiatric disorders to acute symptoms of psychiatric illness in a third dimension.

28. The software method of claim 1, wherein the step of addressing the decline and identifying potential contributing factors, and developing a care plan includes the step of the software automatically listing the information from the defining the decline step and connecting the potential contributing factors to the decline step for treatment.

29. The software method of claim 28, wherein the step of addressing the decline and identifying potential contributing factors, and developing a care plan includes the step of the software prompting the user to set up the goal, objectives, interventions, and care plan orders.

30. The software method of claim 29, wherein the step of addressing the decline and identifying potential contributing factors, and developing a care plan includes the step of developing a monitoring flow sheet for the targeted declines that the user wants to monitor by prompting the user to input specific data to complete the monitoring form.

31. The software method of claim 30, wherein the step of ensuring adequate management of medical and medication factors includes the software prompting the user to input the data from the monitoring flowsheets to evaluate what to do next.

32. The software method of claim 1, wherein the step of addressing psychotropic management includes the step of the software providing the user with the option of using a psychotropic algorithm for the treatment of behavioral problems related to dementia.

33. The software method of claim 32, wherein the step of the software providing the user with the option of using a psychotropic algorithm for the treatment of behavioral problems related to dementia further includes the step of prompting the user to decide which psychiatric symptom group is driving the behavioral decline.

34. The software method of claim 33, wherein the step of the software providing the user with the option of using a psychotropic algorithm for the treatment of behavioral problems related to dementia automatically takes the user to the appropriate part of the psychotropic management algorithm for help on determining the safest and most effective psychotropic.
